(19) United States
(12) Patent Application Publication
(10) Pub. No.: US 2002/0136712 A1
(43) Pub. Date: Sep. 26, 2002

(54) Bacterial phage associated lysing enzymes for the prophylactic and therapeutic treatment of colonization and infections caused by streptococcus pneumoniae

(76) Inventors: Fischetti Vincent, West Hempstead, NY (US); Loeffler Jutta, New York, NY (US); Nelson Daniel, New York, NY (US); Lawrence Loomis, Columbia, MD (US)

Correspondence Address: Jonathan E. Grant, Grant Patent Services, Suite 210, 2120 L Street, N. W., Washington, DC, 20037, US

(21) Appl. No.: 09/960,472

(22) Filed: Sep. 21, 2001

Related U.S. Application Data
(63) Continuation-in-part of application No. 09/846,688, filed on May 2, 2001.
Application No. 09/846,688 is a continuation-in-part of application No. 09/497,495, filed on Apr. 18, 2000.
Application No. 09/497,495 is a continuation of application No. 09/395,636, filed on Sep. 14, 1999.
Application No. 09/395,636 is a continuation-in-part of application No. 08/962,523, filed on Oct. 31, 1997.

(51) Int. Cl. A61K 38/43; C12N 15/86; C12P 21/02; C12N 1/21

(52) U.S. Cl. 424/94.1; 435/456; 435/69.1; 435/252.33

(57) ABSTRACT

A method for the prophylactic and therapeutic treatment of Streptococcal pneumoniae infections is disclosed which comprises the treating of an individual with an effective amount of a lytic enzyme composition specific for the infecting bacteria, and a carrier for delivering said lytic enzyme. This method, and composition can be used for the treatment of upper respiratory infections, lower respiratory infections, septicemia, bacterial meningitis, and other infections involving Streptococcal pneumoniae.

The following application is a continuation-in-part of U.S. patent application Ser. No. 09/846,688, filed May 2, 2001, which is a continuation in part of Ser. No. 09/497,495, filed Apr. 18, 2000, now U.S. Pat. No. 6,238,661, which was a continuation of U.S. patent application Ser. No. 09/395,636, filed Sep. 14,1999, now U.S. Pat. No. 6,056,954, issued May 2, 2000, which was a continuation in part of U.S. patent application Ser. No. 08/962,523, filed Oct. 31, 1997, now U.S. Pat. No. 5,997,862.

### DESCRIPTION

### BACKGROUND OF THE INVENTION

1. Field of the Invention

The present invention relates to methods and compositions for the prophylaxis and treatment of Streptococcus pneumoniae.

2. Description of the Prior Art

In the past, antibiotics have been used to treat various infections. The work of Selman Waksman in the introduction and production of Streptomycetes, and Dr. Fleming's discovery of penicillin, as well as the work of numerous others in the field of antibiotics, are well known. Over the years, there have been additions and chemical modifications to the “basic” antibiotics to make them more powerful, or to treat people allergic to these antibiotics.

Others have found new uses for these antibiotics. U.S. Pat. No. 5,260,292 (Robinson et al.) discloses the topical treatment of acne with aminopenicillins. The method and composition for topically treating acne and acneiform dermal disorders includes applying an antibiotic selected from the group consisting of ampicillin, amoxicillin, other aminopenicillins, and cephalosporins, and derivatives and analogs thereof, effective to treat the acne and acneiform dermal disorders. U.S. Pat. No. 5,409,917 (Robinson et al.) discloses the topical treatment of acne with cephalosporins.

However, as more antibiotics have been prescribed or used at an ever increasing rate for a variety of illnesses, increasing numbers ofbacteria have developed a resistance to antibiotics. Larger doses of stronger antibiotics are now being used to treat ever more resistant strains of bacteria. Multiple antibiotic resistant bacteria have consequently developed. The use of more antibiotics and the number of bacteria showing resistance has led to increasing the amount of time that the antibiotics need to be used. Broad, non-specific antibiotics, some of which have detrimental effects on the patient, are now being used more frequently. Also, antibiotics do not easily penetrate mucus linings. Additionally, the number of people allergic to antibiotics appears to be increasing. Consequently, other efforts have been sought to first identify and then kill bacteria.

Attempts have been made to treat bacterial diseases with the use of bacteriophages. U.S. Pat. No. 5,688,501 (Merril, et al.) discloses a method for treating an infectious disease caused by bacteria in an animal with lytic or non-lytic bacteriophages that are specific for particular bacteria.

U.S. Pat. No. 4,957,686 (Norris) discloses a procedure of improved dental hygiene which comprises introducing into the mouth bacteriophages parasitic to bacteria which possess the property of readily adhering to the salivary pellicle.

It is to be noted that the direct introduction of bacteriophages into an animal to prevent or fight diseases has certain drawbacks. Specifically, the bacteria must be in the right growth phase for the phage to attach. Both the bacteria and the phage have to be in the correct and synchronized growth cycles. Additionally, there must be the right number of phages to attach to the bacteria; if there are too many or too few phages, there will be either no attachment or no production of the lysing enzyme. The phage must also be active enough. The phages are also inhibited by many things including bacterial debris from the organism it is going to attack. Further complicating the direct use of a bacteriophage to treat bacterial infections is the possibility of immunological reactions, rendering the phage non-functional.

Consequently, others have explored the use of other safer and more effective means to treat and prevent bacterial infections.

U.S. Pat. No. 5,604,109 (Fischetti et al.) relates to the rapid detection of Group A streptococci in clinical specimens, through the enzymatic digestion by a semi-purified Group C streptococcal phage associated lysin enzyme. This enzyme work became the basis of additional research, leading to methods of treating diseases.

U.S. Pat. No. 5,985,271 (Fischetti and Loomis) and U.S. Pat. No. 6,017,528 (Fischetti and Loomis) disclose the use of an oral delivery mode, such as a candy, chewing gum, lozenge, troche, tablet, a powder, an aerosol, a liquid or a liquid spray, containing a lysin enzyme produced by group C streptococcal bacteria infected with a Cl bacteriophage for the prophylactic and therapeutic treatment of Streptococcal A throat infections, commonly known as strep throat.

U.S. Pat. No. 6,056,954 (Fischetti and Loomis) discloses a method for the prophylactic and therapeutic treatment of bacterial infections of the skin, vagina, or eyes which comprises the treatment of an individual with an effective amount of a lytic enzyme composition specific for the infecting bacteria, wherein the lytic enzyme is in an environment having a pH which allows for activity of said lytic enzyme; and a carrier for delivering said lytic enzyme.

U.S. Pat. No. 6,056,955 (Fischetti and Loomis) discloses a method and composition for the topical treatment of streptococcal infections by the use of a lysin enzyme blended with a carrier suitable for topical application to dermal tissues. The method for the treatment of dermatological streptococcal infections comprises administering a composition comprising effective amount of a therapeutic agent, with the therapeutic agent comprising a lysin enzyme produced by group C streptococcal bacteria infected with a Cl bacteriophage. The therapeutic agent can be in a pharmaceutically acceptable carrier.

U.S. Pat. No. 6,238,661 (Fischetti and Loomis) discloses a method for the prophylactic and therapeutic treatment of bacterial infections in general, which comprise administering to an individual an effective amount of a composition comprising an effective amount of lytic enzyme and a carrier for delivering the lytic enzyme and the method of treating illnesses in general.

U.S. Pat. No. 6,248,324 (Fischetti and Loomis) discloses a composition for dermatological infections by the use of a lytic enzyme in a carrier suitable for topical application to dermal tissues. The method for the treatment of dermatological infections comprises administering a composition comprising an effective amount of a therapeutic agent, with the therapeutic agent comprising a lytic enzyme produced by infecting a bacteria with phage specific for that bacteria.

U.S. Pat. No. 6,254,866 (Fischetti and Loomis) discloses a method for treatment of bacterial infections of the digestive tract which comprises administering a lytic enzyme specific for the infecting bacteria. The lytic enzyme is preferably in a carrier for delivering the lytic enzyme. The bacteria to be treated is selected from the group consisting of Listeria, Salmonella, E. coli, Campylobacter, and combinations thereof. The carrier for delivering at least one lytic enzyme to the digestive tract is selected from the group consisting of suppository enemas, syrups, or enteric coated pills.

U.S. Pat. No. 6,264,945 (Fischetti and Loomis) discloses a method and composition for the treatment of bacterial infections by the parenteral introduction of at least one lytic enzyme produced by a bacteria infected with a bacteriophage specific for that bacteria and an appropriate carrier for delivering the lytic enzyme into a patient. The injection can be done intramuscularly, subcutaneously, or intravenously.

### SUMMARY OF THE INVENTION

Methods for obtaining and purifying bacteriaphage lytic enzymes produced by bacteria infected with bacteriophage are known in the art. Recent evidence suggests that the phage enzyme that lyses the streptococcus organism may in limited cases actually be a bacterial enzyme that is used to construct the bacterial cell wall. While replicating in the bacterium, a phage gene product may cause the upregulation or derepression of bacterial enzyme for the purpose of releasing the bacteriophage. These bacterial enzymes may be tightly regulated by the bacterial cell and are used by the bacteria for the construction and assembly of the cell wall. In general, however, phage lytic enzymes are coded for by the phage genome and produced by the phage in the infected bacterial host for phage release.

In this context of course, the term “lytic enzyme genetically coded for by a bacteriophage” means a polypeptide having at least some lytic activity against the host bacteria. The polypeptide has a sequence that encompasses native sequence lytic enzyme and variants thereof. The polypeptide may be isolated from a variety of sources, such as from phage, as emphasized in this specification due to convenience, or prepared by recombinant or synthetic methods, as emphasized in the cited research, such as those by Garcia et al. Every polypeptide has two domains, a choline binding portion at the carboxyl terminal side and a amidase activity that acts upon amide bonds in the peptidoglycan at the amino terminal side. Generally speaking a lytic enzyme according to the invention is between 25,000 and 35,000 daltons in molecular weight and comprises a single polypeptide chain; however, this can vary depending on the enzyme chain. The molecular weight most conveniently is determined by assay on denaturing sodium dodecyl sulfate gel electrophoresis and comparison with molecular weight markers.

It should be understood that bacteriophage lytic enzyme are enzymes that specifically cleave bonds that are present in the peptidoglycan of bacterial cells. Since the bacterial cell wall peptidoglycan is highly conserved among all bacteria, there are only a few bonds to be cleaved to disrupt the cell wall. Enzymes that cleave these bonds are muramidases, glucosaminidases, endopeptidases, or N-acetyl-muramoyl-L-alanine amidases (hereinafter referred to as amidases). The majority of reported phage enzymes are either muramidases or amidases, and there have been no reports of bacteriophage glucosaminidases. Fischetti et al (1974) reported that the Cl streptococcal phage lysin enzyme was an amidase. Garcia et al (1987, 1990) reported that the Cpl lysin from a S. pneumoniae from a Cp-1 phage was a lysozyme. Caldentey and Bamford (1992) reported that a lytic enzyme from the phi 6 Pseudomonas phage was an endopeptidase, splitting the peptide bridge formed by melo-diaminopimilic acid and D-alanine. The E. coli T1 and T6 phage lytic enzymes are amidases as is the lytic enzyme from Listeria phage (ply) (Loessner et al, 1996). There are also other enzymes which cleave the cell wall.

Embodiment of the invention concerns the extraction and use of a bacterial phage associated lytic enzymes for the treatment and prevention of Streptococcus pneumoniae, also referred to as pneumococcus. In one such embodiment the bacterial phage associated lytic enzyme is prepared by growing up phage in an infected bacterium and harvesting the enzyme. In another such embodiment the bacterial phage associated lytic enzyme is prepared recombinantly by growing a transgenic bacterium that makes the enzyme and extracting the enzyme from the bacterium.

“A native sequence phage associated lytic enzyme” is a polypeptide having the same amino acid sequence as an enzyme derived from nature. Such native sequence enzyme can be isolated from nature or can be produced by recombinant or synthetic means. The term “native sequence enzyme” specifically encompasses naturally occurring forms (e.g., alternatively spliced or modified forms) and naturally-occurring variants of the enzyme. In one embodiment of the invention, the native sequence enzyme is a mature or full-length polypeptide that is genetically coded for by a gene from a bacteriophage specific for Streptococcus pneumoniae. Of course, a number of variants are possible and known, as acknowledged in publications such as Lopez et al., Microbial Drug Resistance 3: 199-211 (1997); Garcia et al., Gene 86: 81-88 (1990); Garcia et al., Proc. Natl. Acad. Sci. USA 85: 914-918 (1988); Garcia et al., Proc. Natl. Acad. Sci. USA 85: 914-918 (1988); Garcia et al., Streptococcal Genetics (J. J. Ferretti and Curtis eds., 1987); Lopez et al., FEMS Microbiol. Lett. 100: 439-448 (1992); Romero et al., J. Bacteriol. 172: 5064-5070 (1990); Ronda et al., Eur. J. Biochem. 164: 621-624 (1987) and Sanchez et al., Gene 61: 13-19 (1987). The contents of each of these references, particularly the sequence listings and associated text that compares the sequences, including statements about sequence homologies, are specifically incorporated by reference in their entireties.

“A variant sequence phage associated lytic enzyme” means a functionally active lytic enzyme genetically coded for by a bacteriophage specific for Streptococcus pneumoniae, as defined below having at least about 80% amino acid sequence identity with the sequence shown as SEQ ID No. 1. Such phage associated lytic enzyme variants include, for instance, lytic enzyme polypeptides wherein one or more amino acid residues are added, or deleted at the N or C terminus of the sequence of SEQ ID No. 1. Ordinarily a phage associated lytic enzyme will have at least about 80% or 85% amino acid sequence identity with native phage associated lytic enzyme sequences, more preferably at least about 90% (e.g. 90%) amino acid sequence identity. Most preferably a phage associated lytic enzyme variant will have at least about 95% (e.g. 95%) amino acid sequence identity with the native phage associated lytic enzyme of SEQ ID No. 1.

“Percent amino acid sequence identity” with respect to the phage associated lytic enzyme sequences identified herein is defined as the percentage of amino acid residues in a candidate sequence that are identical with the amino acid residues in the phage associated lytic enzyme sequence, after aligning the sequences in the same reading frame and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, such as using publicly available computer software such as blast software. Those skilled in the art can determine appropriate parameters for measuring alignment, including any algorithms needed to achieve maximal alignment over the whole length of the sequences being compared.

“Percent nucleic acid sequence identity” with respect to the phage associated lytic enzyme sequences identified herein is defined as the percentage of nucleotides in a candidate sequence that are identical with the nucleotides in the phage associated lytic enzyme sequence, after aligning the sequences and introducing gaps, if necessary, to achieve the maximum percent sequence identity. Alignment for purposes of determining percent nucleic acid sequence identity can be achieved in various ways that are within the skill in the art, including but not limited to the use of publically available computer software. Those skilled in the art can determine appropriate parameters for measuring alignment, including any algorithms needed to achieve maximal alignment over the full length of the sequences being compared.

“Polypeptide” refers to a molecule comprised of amino acids which correspond to those encoded by a polynucleotide sequence which is naturally occurring. The polypeptide may include conservative substitutions where the naturally occurring amino acid is replaced by one having similar properties, where such conservative substitutions do not alter the function of the polypeptide (see, for example, Lewin “Genes V” Oxford University Press Chapter 1, pp. 9-13 1994).

A large variety of isolated cDNA sequences that encode phage associated lysing enzymes and partial sequences that hybridize with such gene sequences are useful for recombinant production of the lysing enzyme. Representative nucleic acid sequences in this context are SEQ ID No. 2 sequence shown in FIG. 6 and sequences that hybridize with complementary sequences of a DNA having a sequence shown in FIG. 6 under stringent conditions. Still further variants of these sequences and sequences of nucleic acids that hybridize with those shown in the figures also are contemplated for use in production of lysing enzymes according to the invention, including natural variants that may be obtained.

Many of the contemplated variant DNA molecules include those created by standard DNA mutagenesis techniques, for example, M13 primer mutagenesis. Details of these techniques are provided in Sambrook et al. (1989) In Molecular Cloning: A Laboratory Manual, Cold Spring Harbor, N.Y. (incorporated herein by reference). By the use of such techniques, variants may be created which differ in minor ways from those disclosed. DNA molecules and nucleotide sequences which are derivatives of those specifically disclosed herein and which differ from those disclosed by the deletion, addition or substitution of nucleotides while still encoding a protein which possesses the functional characteristic of the BSMR protein are contemplated by this invention. Also within the scope of this invention are small DNA molecules which are derived from the disclosed DNA molecules. Such small DNA molecules include oligonucleotides suitable for use as hybridization probes or polymerase chain reaction (PCR) primers. As such, these small DNA molecules will comprise at least a segment of a lytic enzyme genetically coded for by a bacteriophage specific for Streptococcus pneumoniae and, for the purposes of PCR, will comprise at least a 10-15 nucleotide sequence and, more preferably, a 15-30 nucleotide sequence of the gene. DNA molecules and nucleotide sequences which are derived from the disclosed DNA molecules as described above may also be defined as DNA sequences which hybridize under stringent conditions to the DNA sequences disclosed, or fragments thereof.

Hybridization conditions corresponding to particular degrees of stringency vary depending upon the nature of the hybridization method of choice and the composition and length of the hybridizing DNA used. Generally, the temperature of hybridization and the ionic strength (especially the sodium ion concentration) of the hybridization buffer will determine the stringency of hybridization. Calculations regarding hybridization conditions required for attaining particular degrees of stringency are discussed by Sambrook et al. (1989), In Molecular Cloning: A Laboratory Manual, Cold Spring Harbor, N.Y., chapters 9 and 11, (herein incorporated by reference).

An example of such calculation is as follows. A hybridization experiment may be performed by hybridization of a DNA molecule (for example, a natural variation of the lytic enzyme genetically coded for by a bacteriophage specific for Streptococcus pneumoniae) to a target DNA molecule. A target DNA may be, for example, the corresponding cDNA which has been electrophoresed in an agarose gel and transferred to a nitrocellulose membrane by Southern blotting (Southern (1975). J. Mol. Biol. 98:503), a technique well known in the art and described in Sambrook et al. (1989) In Molecular Cloning: A Laboratory Manual, Cold Spring Harbor, N.Y. (incorporated herein by reference). Hybridization with a target probe labeled with isotopic P (32) labelled-dCTP is carried out in a solution of high ionic strength such as 6 times SSC at a temperature that is 20-25 degrees Celsius below the melting temperature, Tm, (described infra). For such Southern hybridization experiments where the target DNA molecule on the Southern blot contains 10 ng of DNA or more, hybridization is carried out for 6-8 hours using 1-2 ng/ml radiolabeled probe (of specific activity equal to 109 CPM/mug or greater). Following hybridization, the nitrocellulose filter is washed to remove background hybridization. The washing conditions are as stringent as possible to remove background hybridization while retaining a specific hybridization signal. The term “Tm” represents the temperature above which, under the prevailing ionic conditions, the radiolabeled probe molecule will not hybridize to its target DNA molecule.

The Tm of such a hybrid molecule may be estimated from the following equation: Tm=81.5 degrees C.-16.6(log 10 of sodium ion concentration)+0.41 (%G+C)-0.63(% formamide)-(600/l) where l=the length of the hybrid in base pairs. This equation is valid for concentrations of sodium ion in the range of 0.01M to 0.4M, and it is less accurate for calculations of Tm in solutions of higher sodium ion concentration (Bolton and McCarthy (1962). Proc. Natl. Acad. Sci. USA 48:1390) (incorporated herein by reference). The equation also is valid for DNA having G+C contents within 30% to 75%, and also applies to hybrids greater than 100 nucleotides in length. The behavior of oligonucleotide probes is described in detail in Ch. 11 of Sambrook et al. (1989). In Molecular Cloning: A Laboratory Manual, Cold Spring Harbor, N.Y. (incorporated herein by reference). The preferred exemplified conditions described here are particularly contemplated for use in selecting variations of the lytic gene.

Thus, by way of example, of a 150 base pair DNA probe derived from the first 150 base pairs of the open reading frame of a cDNA having a % GC=45%, a calculation of hybridization conditions required to give particular stringencies may be made as follows:

Assuming that the filter will be washed in 0.3× SSC solution following hybridization, sodium ion=0.045M; % GC=45%; Formamide concentration=0 l=150 base pairs (see equation in Sambrook et al.) and so Tm=74.4 degrees C. The Tm of double-stranded DNA decreases by 1-1.5 degrees C. with every 1% decrease in homology (Bonner et al. (1973). J. Mol. Biol. 81:123). Therefore, for this given example, washing the filter in 0.3 times SSC at 59.4-64.4 degrees C. will produce a stringency of hybridization equivalent to 90%; DNA molecules with more than 10% sequence variation relative to the target BSMR cDNA will not hybridize. Alternatively, washing the hybridized filter in 0.3 times SSC at a temperature of 65.4-68.4 degrees C. will yield a hybridization stringency of 94%; DNA molecules with more than 6% sequence variation relative to the target BSMR cDNA molecule will not hybridize. The above example is given entirely by way of theoretical illustration. One skilled in the art will appreciate that other hybridization techniques may be utilized and that variations in experimental conditions will necessitate alternative calculations for stringency.

In preferred embodiments of the present invention, stringent conditions may be defined as those under which DNA molecules with more than 25% sequence variation (also termed “mismatch”) will not hybridize. In a more preferred embodiment, stringent conditions are those under which DNA molecules with more than 15% mismatch will not hybridize, and more preferably still, stringent conditions are those under which DNA sequences with more than 10% mismatch will not hybridize. In a most preferred embodiment, stringent conditions are those under which DNA sequences with more than 6% mismatch will not hybridize.

The degeneracy of the genetic code further widens the scope of the present invention as it enables major variations in the nucleotide sequence of a DNA molecule while maintaining the amino acid sequence of the encoded protein. For example, a representative amino acid residue is alanine. This may be encoded in the cDNA by the nucleotide codon triplet GCT. Because of the degeneracy of the genetic code, three other nucleotide codon triplets—GCT, GCC and GCA—also code for alanine. Thus, the nucleotide sequence of the gene could be changed at this position to any of these three codons without affecting the amino acid composition of the encoded protein or the characteristics of the protein. The genetic code and variations in nucleotide codons for particular amino acids are well known to the skilled artisan. Based upon the degeneracy of the genetic code, variant DNA molecules may be derived from the cDNA molecules disclosed herein using standard DNA mutagenesis techniques as described above, or by synthesis of DNA sequences. DNA sequences which do not hybridize under stringent conditions to the cDNA sequences disclosed by virtue of sequence variation based on the degeneracy of the genetic code are herein comprehended by this invention.

One skilled in the art will recognize that the DNA mutagenesis techniques described here can produce a wide variety of DNA molecules that code for a bacteriophage lysin specific for Streptococcus pneumoniae yet that maintain the essential characteristics of the lytic protein. Newly derived proteins may also be selected in order to obtain variations on the characteristic of the lytic protein, as will be more fully described below. Such derivatives include those with variations in amino acid sequence including minor deletions, additions and substitutions.

While the site for introducing an amino acid sequence variation is predetermined, the mutation per se does not need to be predetermined. For example, in order to optimize the performance of a mutation at a given site, random mutagenesis may be conducted at the target codon or region and the expressed protein variants screened for the optimal combination of desired activity. Techniques for making substitution mutations at predetermined sites in DNA having a known sequence as described above are well known.

Amino acid substitutions are typically of single residues; insertions usually will be on the order of about from 1 to 10 amino acid residues; and deletions will range about from 1 to 30 residues. Deletions or insertions preferably are made in adjacent pairs, i.e., a deletion of 2 residues or insertion of 2 residues. Substitutions, deletions, insertions or any combination thereof may be combined to arrive at a final construct. Obviously, the mutations that are made in the DNA encoding the protein must not place the sequence out of reading frame and preferably will not create complementary regions that could produce secondary MRNA structure (EP 75,444A).

Substitutional variants are those in which at least one residue in the amino acid sequence has been removed and a different residue inserted in its place. Such substitutions generally are made in accordance with the following Table 1 when it is desired to finely modulate the characteristics of the protein. Table 1 shows amino acids which may be substituted for an original amino acid in a protein and which are regarded as conservative substitutions.

Table 1

| !Original Residue? ! Conservative Substitutions | |
|---|---|
| Ala | ser |
| Arg | lys |
| Asn | gln, his |
| Asp | glu |
| Cys | ser |
| Gln | asn |
| Glu | asp |
| Gly | pro |
| His | asn; gln |
| Ile | leu, val |
| Leu | ile; val |
| Lys | arg; gln; glu |
| Met | leu; ile |
| Phe | met; ieu; tyr |
| Ser | thr |
| Thr | ser |
| Trp | tyr |
| Tyr | trp;phe |
| Val | ile; leu |

Substantial changes in function or immunological identity are made by selecting substitutions that are less conservative than in Table 1, i.e., selecting residues that differ more significantly in their effect on maintaining: (a) the structure of the polypeptide backbone in the area of the substitution, for example, as a sheet or helical conformation; (b) the charge or hydrophobicity of the molecule at the target site; or (c) the bulk of the side chain. The substitutions which in general are expected to produce the greatest changes in protein properties will be those in which: (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histadyl, is substituted for (or by) an electronegative residue, e.g., glutamyl or aspartyl; or (d) a residue having a bulky side chain, e.g., phenylalanine, is substituted for (or by) one not having a side chain, e.g., glycine.

The effects of these amino acid substitutions or deletions or additions may be assessed for derivatives of the lytic protein by analyzing the ability of the derivative proteins to complement the sensitivity to DNA cross-linking agents exhibited by phages in infected bacteria hosts. These assays may be performed by transfecting DNA molecules encoding the derivative proteins into the bacteria as described above.

Having herein provided nucleotide sequences that code for lytic enzyme genetically coded for by a bacteriophage specific for Streptococcus pneumoniae and fragments of that enzyme, correspondingly provided are the complementary DNA strands of the cDNA molecule and DNA molecules which hybridize under stringent conditions to the lytic enzyme cDNA molecule or its complementary strand. Such hybridizing molecules include DNA molecules differing only by minor sequence changes, including nucleotide substitutions, deletions and additions. Also contemplated by this invention are isolated oligonucleotides comprising at least a segment of the cDNA molecule or its complementary strand, such as oligonucleotides which may be employed as effective DNA hybridization probes or primers useful in the polymerase chain reaction. Hybridizing DNA molecules and variants on the lytic enzyme cDNA may readily be created by standard molecular biology techniques.

The detection of specific DNA mutations may be achieved by methods such as hybridization using specific oligonucleotides (Wallace et al. (1986). Cold Spring Harbor Symp. Quant. Biol. 51:257-261), direct DNA sequencing (Church and Gilbert (1988). Proc. Natl. Acad. Sci. USA 81:1991-1995), the use of restriction enzymes (Flavell et al. (1978). Cell 15:25), discrimination on the basis of electrophoretic mobility in gels with denaturing reagent (Myers and Maniatis (1986). Cold Spring Harbor Symp. Quant. Biol. 51:275-284), RNase protection (Myers et al. (1985). Science 230:1242), chemical cleavage (Cotton et al. (1985). Proc. Natl. Acad. Sci. USA 85:4397-4401) (incorporated herein by reference), and the ligase-mediated detection procedure (Landegren et al., 1988).

Oligonucleotides specific to normal or mutant sequences are chemically synthesized using commercially available machines, labeled radioactively with isotopes (such as .sup.32 P) or non-radioactively (with tags such as biotin (Ward and Langer et al. Proc. Natl. Acad. Sci. USA 78:6633-6657 1981) (incorporated herein by reference), and hybridized to individual DNA samples immobilized on membranes or other solid supports by dot-blot or transfer from gels after electrophoresis. The presence or absence of these specific sequences are visualized by methods such as autoradiography or fluorometric or colorimetric reactions (Gebeyehu et al. Nucleic Acids Res. 15:4513-4534 1987) (incorporated herein by reference).

Sequence differences between normal and mutant forms of the gene may also be revealed by the direct DNA sequencing method of Church and Gilbert (1988) (incorporated herein by reference). Cloned DNA segments may be used as probes to detect specific DNA segments. The sensitivity of this method is greatly enhanced when combined with PCR (Stoflet et al. Science 239:491-494, 1988) (incorporated herein by reference). In this approach, a sequencing primer which lies within the amplified sequence is used with double-stranded PCR product or single-stranded template generated by a modified PCR. The sequence determination is performed by conventional procedures with radiolabeled nucleotides or by automatic sequencing procedures with fluorescent tags. Such sequences are useful for production of lytic enzymes according to embodiments of the invention.

Nasopharyngeal carriage is the major reservoir for Streptococcus pneumoniae in the community and is the source of infections with these organisms. While eliminating this reservoir would impact greatly on disease, no intervention other than antibiotics has been available for this purpose. Streptococcus pneumoniae remains one of the most challenging human pathogens, because of the morbidity and mortality it causes in young children, the elderly and in immunocompromised patients. S. pneumoniae is found in the nasopharynx of 11-76% of the population, averaging 40-50% for children and 20-30% for adults (F. Ghaffar, I. R. Friedland, G. H. McCracken, Jr., Pediatr Infect Dis J 18, 638-46. (1999), incorporated by reference). The asymptomatic carrier state, particularly in children, is thought to be the major reservoir of the pathogen, which is transmitted by salivary aerosols and direct contact. Under predisposing conditions, such as a concomitant viral infection, the organism will spread locally or systemically.

Pneumococci account for the majority of cases of acute otitis media (AOM), community acquired pneumoniae and bacterial meningitis, and can cause lethal sepsis. In recent years, resistance of pneumococci to multiple antibiotics has increased worldwide. Many studies have shown that treatment with antibiotics in children, be it for AOM or eradication of group A streptococci, even with a single dose, is associated with an increase in the carriage of resistant pneumococcal strains (E. Melander, et al., Eur J Clin Microbiol Infect Dis 17, 834-8. (1998), T. Heikkinen, et al., Acta Paediatr 89, 1316-21. (2000), and J. Y. Morita, et al., Pediatr Infect Dis J 19, 41-6. (2000), all incorporated by referencen). Treatment of pneumococcal disease is thus becoming more difficult than in the past. The number of annual cases of AOM in the United States is about 7 million, while invasive pneumococcal infection was recently estimated to be more than 60,000 with an overall mortality of 10%. Although most of these latter cases occurred in persons eligible for vaccination (K. A. Robinson, et al., JAMA 285, 1729-35. (2001), incorporated by reference.), vaccination rates remain insufficient (C. G. Stevenson, M. A. McArthur, M. Naus, E. Abraham, A. J. McGeer, CMAJ 164,1413-9. (2001), S. Gleich, et al., Infect Control Hosp Epidemiol 21, 711-7. (2000) incorporated by reference). Furthermore, despite the progress that has been made with the development of conjugate vaccines for children younger than 2 years, it remains doubtful that vaccination alone is sufficient to eliminate carriage of and disease caused by pneumococci. The new conjugate vaccines include a restricted number of pneumococcal serotypes and protect only incompletely against colonization with these. About one third to one half of cases of AOM are caused by strains not included in a 9-valent vaccine (S. I. Pelton, Vaccine 19 Suppl 1, S96-9. (2000), incorporated by reference). Moreover, an increase in the carriage of non-vaccine serotypes has been reported (N. Mbelle, et al., J Infect Dis 180, 1171-6. (1999), incorporated by reference). Because of these problems, there is a need for an alternative preventive strategy for situations where vaccination is insufficient, impossible or inefficient.

Eradication or even reduction of nasopharyngeal carriage likely will impact on the transmission of S. pneumoniae and the incidence of infection. Antibiotic prophylaxis in controlled surroundings has shown limited success but carries the risk of selective pressure resulting in an increase of resistant strains (S. D. Putnam, G. C. Gray, D. J. Biedenbach, R. N. Jones, Clin Microbiol Infect 6, 2-8. (2000). incorporated by reference). Until now, there has been no substance that can specifically reduce the number of pneumococci carried on human mucous membranes without affecting the normal indigenous mucosal flora.

By using the present enzyme of the present invention, a purified pneumococcal bacteriophage lytic enzyme (Pal) is able to kill 15 common serotypes of pneumococci, including penicillin-resistant strains. However, this enzyme is specific for pneumococci; the Pal enzyme has little to no effect on bacterial flora normally found in the human oropharynx.

The use of phage associated lytic enzymes produced by the infection of a bacteria with a bacteria specific phage has numerous advantages for the treatment of diseases. As the phage are targeted for specific bacteria, the lytic enzymes do not interfere with normal flora. Also, lytic phages primarily attack cell wall structures which are not affected by plasmid variation. The actions of the lytic enzymes are fast. Yet another advantage is that the phage associated lytic enzymes can be produced by a natural process (infection of bacteria with phage) or by a synthetic process such as by recombinant means.

It is an object of the invention to use phage associated enzymes to prophylactically and therapeutically treat bacterial diseases.

The invention (which incorporates U.S. Pat. No. 5,604,109 in its entirety by reference) uses a lytic enzyme genetically coded for by a bacteriophage specific for Streptococcus pneumoniae as a prophylactic treatment for eliminating or reducing the carriage of pneumococci, preventing those who have been exposed to others who have the symptoms of an infection from getting sick, or as a therapeutic treatment for those who have already become ill from the infection. The present invention is based upon the discovery that phage lytic enzymes specific for bacteria infected with a specific phage can effectively and efficiently break down the cell wall of the bacterium in question. At the same time, the semipurified enzyme is lacking in proteolytic enzymatic activity and is therefore is non-destructive to mammalian proteins and tissues when present during the digestion of the bacterial cell wall.

In one embodiment of the invention, a phage associated lytic enzyme is put into a carrier which is placed in an inhaler to treat or prevent the spread of diseases localized in the mucus lining of the oral cavity, lungs, and nasopharynx. The lytic enzymes can be directed to the mucosal lining, where, in residence, they will be able to kill colonizing bacteria. Accordingly, in one embodiment of the invention, a phage enzyme, and/or its peptide fragments are directed to the mucosal lining, where, in residence, they kill colonizing disease bacteria.

In another embodiment of the invention a lytic enzyme is administered in the form of a candy, chewing gum, lozenge, troche, tablet, a powder, an aerosol, a liquid, a liquid spray, or toothpaste for the prevention or treatment of bacterial infections associated with upper respiratory tract illnesses.

Similarly, the lytic enzyme can be used to treat lower respiratory tract illnesses, particularly by the use of bronchial sprays intravenous administration of the enzyme.

In another embodiment of the invention, a lytic enzyme is administered to the ear of a patient.

In yet another embodiment of the invention, a lytic enzyme is administered parenterally, wherein the phage associated lytic enzyme is administered intramuscularly, intrathecally, subdermally, subcutaneously, or intravenously to treat infections by Streptococcus pneumoniae.

This invention may also be used to treat septicemia.

It is another object of the invention to apply a phage associated lytic enzyme intravenously, to treat septicemia and general infections of Streptococcus pneumoniae.

In another embodiment of the invention, a lytic enzyme is applied to the eye to treat an infection of Streptococcus pneumoniae. In one form of this invention, the enzyme is applied by means of eye drops.

In another embodiment of the invention, a lytic enzyme is included in a contact lense cleaning solution to treat or prevent infections by Streptococcus pneumoniae.

In a further embodiment of the invention, conventional antibiotics may be included in the therapeutic agent with the lytic enzyme and with or without the presence of lysostaphin.

In another embodiment of the invention, more than one lytic enzyme may also be included in the therapeutic agent.

While an enzyme can be produced by directly infecting S. pneumoniae with a Dp1 phage or another phage which is specific for a S. pneumoniae, the lytic enzyme may be produced by removing a gene for the lytic enzyme from the phage genome, putting the gene into a transfer vector, and cloning said transfer vector into an expression system.

### BRIEF DESCRIPTION OF THE DRAWINGS

The accompanying drawings, which are incorporated into and constitute part of this specification, illustrate the preferred embodiments of the invention, and together with the detailed description below, serve to explain the invention in greater detail.

FIG. 1. is a SDS-Page analysis of the purified Pal enzyme;

FIG. 2 is a bar graph showing the in vitro killing of 15 clinical S. pneumoniae strains, 2 pneumococcal mutants and 5 oral streptococcal species in log-phase with Pal;

FIG. 3 are electron micrographs of cells of S. pneumoniae as they are exposed to Pal enzyme;

FIG. 4 is a graph showing the elimination of S. pneumoniae serotype 14 in the mouse model of nasopharyngeal carriage;

FIG. 5 is an amino acid sequence listing, SEQ ID No. 1, for the Pal lytic enzyme genetically coded for by a bacteriophage specific for Streptococcus pneumoniae; and

FIG. 6 is a nucleic acid sequence listing, SEQ ID No. 2, for the whole genome of the bacteriophage Dp1, specific for Streptococcus pneumoniae, with bases 3687 to 4577 genetically coding for the Pal lytic enzyme.

### DETAILED DESCRIPTION OF THE INVENTION

One embodiment of the present invention is a method for treating Streptococcus pneumoniae which comprises treating the infection with a therapeutic agent comprising an effective amount of at least one lytic enzyme specific for Streptococcus pneumoniae. More specifically, a lytic enzyme specific for lysing the cell wall of Streptococcus pneumoniae is produced from genetic material from a bacteriophage specific for Streptococcus pneumoniae. The lytic enzyme can be produced in a number of ways. In a preferred embodiment a gene for the lytic enzyme from the phage genome is put into a transfer or movable vector, preferably a plasmid, and the plasmid is cloned into an expression vector or expression system. The expression vector may be E. coli, Bacillus, or a number of other suitable bacteria. The vector system may also be a cell free expression system. All of these methods of expressing a gene or set of genes are known in the art. The lytic enzyme may also be created by infecting Streptococcus pneumoniae with a bacteriophage specific for Streptococcus pneumoniae, wherein said at least one lytic enzyme exclusively lyses the cell wall of said Streptococcus pneumoniae having at most minimal effects on other bacterial flora present.

There are a number of bacteriophages for S. pneumoniae, including but not limited to Dp-1, DP-4, Cp-l, Cp-7, Cp-9, Cp-5, MM1, EJ-1,HB-3, HB-623, HB-746, ω-1, and ω-2. The pneumococcal phages from which the gene for the lytic enzyme is cloned are classified in four groups based on their viral families. All contain double-stranded DNA and a cell wall lytic system consisting of a holin that permeabilizes the cell membrane, and either an N-acetylmuramoyl-L-alanine amidase (amidase) or a lysozyme, capable of digesting the pneumococcal cell wall. (P. Garcia, A.C. Martin, R. Lopez, Microb Drug Resist 3, 165-76 (1997), incorporated by reference). Both types of enzymes contain a C-terminal choline-binding domain common to many pneumococcal proteins and an N-terminal catalytic domain. The lytic system allows the virus to escape the host cell after successful replication.

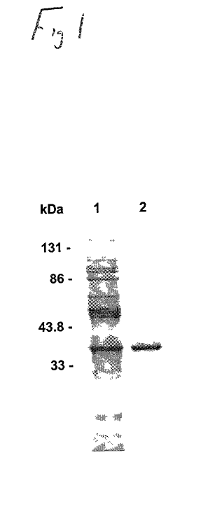

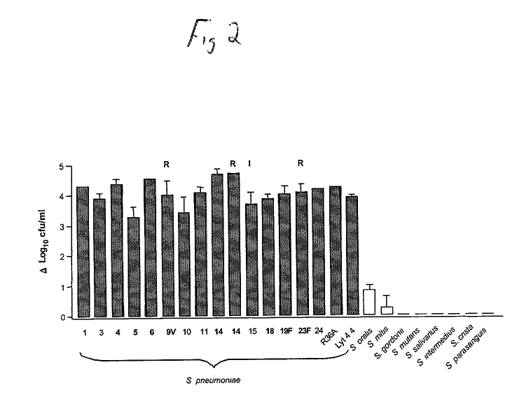

The lytic enzyme first had to be produced to study its possible effectiveness for treating Streptococcus pneumoniae. To do so, E. coli DH5 a (PMSP11) expressing the amidase Pal of phage Dp-1 was obtained from R. Lopez of Center for Biological Investigations, Madrid Spain. See M M Sheehan, J. L. Garcia, R. Lopez, P. Garcia. Mol. Microbiol 25, 717-725 (1997) incorporated herein by reference. The enzyme was produced in E. coli and purified by affinity chromatography in a single step as described, with some modifications, in J. M. Sanchez-Puelles, J. M. Sanz, J. L. Garcia, E. Garcia, Eur J Biochem 203, 153-9. (1992), (incorporated herein by reference). In brief, E. coli were harvested by centrifugation, suspended in enzyme buffer (20 mM phosphate buffer (PB), 1 mM EDTA, 10 mM DTT) and broken by sonication for 1.5 min on ice. The crude extracts were ultracentrifuged (75,000× g for 1 h at 4° C.), the supernatant loaded on a DEAE-cellulose column (volume 20 ml) and washed with 3 volumes of 20 mM PB (pH 7.0), 4 volumes of PB containing 1 M NaCl, and 2 volumes of PB containing 0.1 M NaCl. The enzyme was eluted with PB containing 0.1 M NaCl and 6.5% (w/v) choline. Pooled fractions were dialyzed overnight (1:75) against enzyme buffer. Purification was verified by SDS-PAGE. Protein content was measured with the Bradford method using the dye reagent from Biorad (Hercules, Calif.). FIG. 1 shows the Page analysis 1 of the purified Pal enzyme, with lane 1 showing the crude extract from DH5-alpha, and lane 2 showing the purified Pal after affinity chromatography on DEAE cellulose.

A unit for the enzyme was defined using lysis of exponentially growing S. pneumoniae serogroup 14 with serial dilutions of purified Pal. S. pneumoniae strain DCC 1490 (serotype 14) was grown in a brain heart infusion medium (BHI, Difco Laboratories, Detroit, Mich.) at 37° C. to logarithmic phase, centrifuged at 5000× g for 10 min at 4° C., and resuspended in sterile saline to an absorbance at 600 nm of 1.3. Pal was diluted in an enzyme buffer in serial 2-fold dilutions. In a 96-well plate, 150 ul of the bacterial suspension was incubated with 150 ul of each Pal dilution (150 ul enzyme buffer for the control well). One unit of enzyme was defined as the reciprocal of the dilution, which caused a 50% decrease in absorbance after 15 min incubation at 37° C., as compared with the absorbance of the control well. The purification process yielded an average of 15 U of enzyme per ug protein. (All chemicals were purchased from Sigma (St.Louis, Mo.) unless stated otherwise).

The killing ability of the Pal enzyme in vitro was measured by exposing 15 clinical strains of S. pneumoniae, 2 pneumococcal mutants (R36A, Lyt 4-4) and 5 species of oral commensal streptococci (S. gordonii, S. mitis, S. mutans, S. oralis, S. salivarius) to purified enzyme at a final concentration of 100 U/ml, and in the case of the oral streptococci to 1,000 and 10,000 U/ml (17). The pneumococcal strains, obtained from various sources as shown in Table 2, included 9 serogroups that most frequently cause invasive disease in North America, Europe, Africa and Oceania (W. P. Hausdorff, J. Bryant, P. R. Paradiso, G. R. Siber, Clin. Infect. Dis. 30 100-21 (2000).

TABLE 2
Bacterial strains tested for susceptibility to Pal

| | | Capsular | Susceptibility to | | |
|---|---|---|---|---|---|
| Species | Strain | group/type | Penicillin | Clonal type | Source |
| S. pneumoniae | DCC 1355 | 19F | S | | 1 |
| S. pneumoniae | DCC 1335 | 9V | R | Sp⁹-3 | 1 |
| S. pneumoniae | DCC 1420 | 23F | R | Sp²³-1 | 1 |
| S. pneumoniae | DCC 1476 | 15 | I | | 1 |
| S. pneumoniae | DCC 1490 | 14 | S | | 1 |
| S. pneumoniae | DCC 1494 | 14 | R | Sp¹⁴-1 | 1 |
| S. pneumoniae | DCC 1714 | 3 | S | | 1 |
| S. pneumoniae | DCC 1808 | 24 | S | | 1 |
| S. pneumoniae | DCC 1811 | 11 | S | | 1 |
| S. pneumoniae | DCC 1850 | 6B | S | | 1 |
| S. pneumoniae | AR 314 | 5 | S | | 1 |
| S. pneumoniae | AR 620 | 1 | S | | 1 |
| S. pneumoniae | GB2017 | 18 | S | | 1 |
| S. pneumoniae | GB2092 | 4 | S | | 1 |
| S. pneumoniae | GB2163 | 10 | S | | 1 |
| S. pneumoniae | R36A | | | | 1 |
| S. pneumoniae | Lyt 4-4 | | | | 1 |
| S. gordonii | PK 2565 | | | | 2 |
| S. mitis | J 22 | | | | 2 |
| S. mutans | OMZ 175 | | | | 3 |
| S. oralis | H 1 | | | | 2 |
| S. salivarius | ATCC 27945 | | | | 2 |

R, resistant; I, intermediate; S, susceptible.

1, Alexander Tomasz, The Rockefeller University, New York, N.Y.; 2, Paul Kohlenbrander, National Institute of Dental and Craniofacial Research, Bethesda, Md.; 3, Ivo Van de Rijn, Wake Forest University, Winston-Salem, N.C.

Furthermore, three highly penicillin-resistant strains were included, which represent the internationally spread clones Sp9-3, Spl4-3 and Sp23-1, that account for a majority of penicillin-resistant pneumococci in day care centers and hospitals (R. Sa-Leao, et al., J Infect Dis 182, 1153-60. (2000), R. B. Roberts, A. Tomasz, A. Corso, J. Hargrave, E. Severina, Microb Drug Resist 7, 137-52. (2001), incorporated herein by reference). In 30 seconds, 100 U of Pal decreased the viable titer of the 15 strains of exponentially growing S. pneumoniae by Log₁₀ 4.0 cfu/ml (median, range 3.3-4.7) as compared to controls incubated with the enzyme buffer alone.

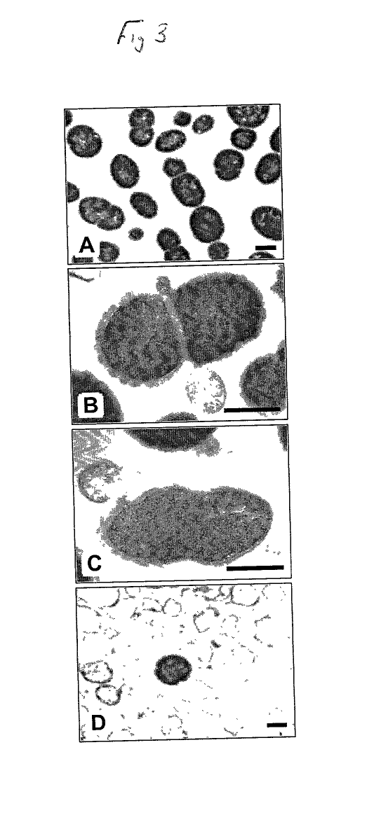

FIG. 2 shows the in vitro killing of 15 clinical S. pneumoniae strains, 2 pneumococcal mutants and 5 oral streptococcal species in log-phase with 100 U/ml Pal during 30 seconds, expressed as the decrease of bacterial titers in powers of 10. Numbers above “S. pneumoniae” indicate serotypes; bold print designates the 9 most frequently isolated serogroups. The error bars show standard deviation of triplicates. I: intermediate susceptibility to penicillin (MIC 0.1-1.0), R: highly penicillin resistant (MIC³ 2.0). Pneumococci with intermediate (n=1) and high penicillin resistance (n=3) were killed at the same rate as penicillin sensitive strains (median (range) Log10 4.0 (3.7-4.7) vs. Log₁₀ 4.1 (3.3-4.7) cfu/ml, p=NS). Moreover, the capsule-deficient laboratory strain R36A and the mutant Lyt 4-4, deficient in a capsule and lacking the major pneumococcal autolysin LytA, showed identical susceptibility to Pal as the clinical pneumococcal isolates (decrease of Log10 4.2 and 3.9 cfu/ml, respectively, p=NS). The latter results suggest that the pneumococcal capsule does not interfere with the enzyme's access to the cell wall and that autolysin does not contribute significantly to cell lysis caused by Pal. One hundred units of Pal also killed exponentially growing S. oralis and S. mitis, but at a significantly lower rate (Log10 0.8 and Log10 0.23 cfu/ml, respectively, p<0.05). Both strains are known to incorporate choline in their cell walls and therefore provide a binding site for the enzyme (S. H. Gillespie, et al. Infect Immun 61, 3076-7 (1993), incorporated by reference). The remaining oral streptococcal strains were unaffected with enzyme concentrations as high as 10,000 U/ml and up to 10 min of exposure.

In vitro, S. pneumoniae, including the R36A and Lyt 4-4 mutants, in stationary phase were more resistant to the lethal action of Pal. Nevertheless, exposure to 10,000 U/ml resulted in killing of Log 10 3.0 cfu/ml (median, range 3.0-4.0) in 30 sec. The mechanism responsible for the decrease in susceptibility to hydrolysis by Pal in non-growing pneumococci is likely to be a change in the cell wall structure (E. I. Tuomanen, A. Tomasz, Scand J Infect Dis Suppl. 74, 102-12 (1991), incorporated herein by reference), such as an increase in peptidoglycan cross-linking.

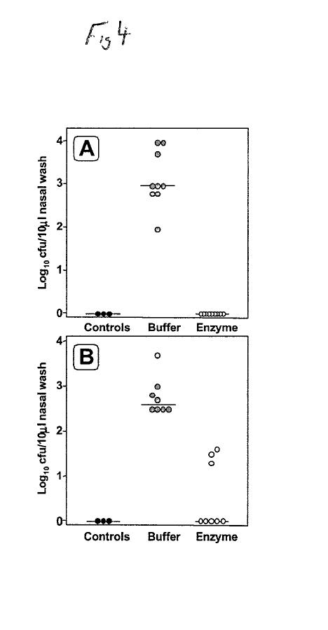

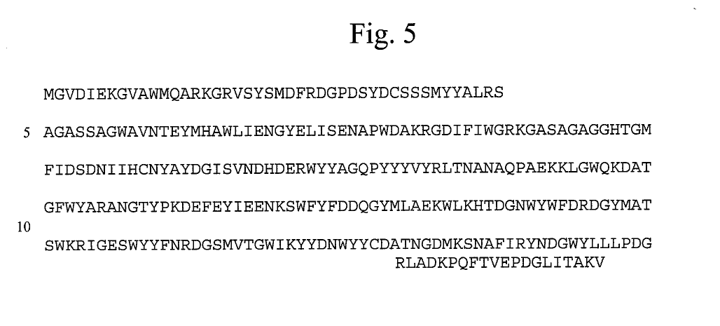

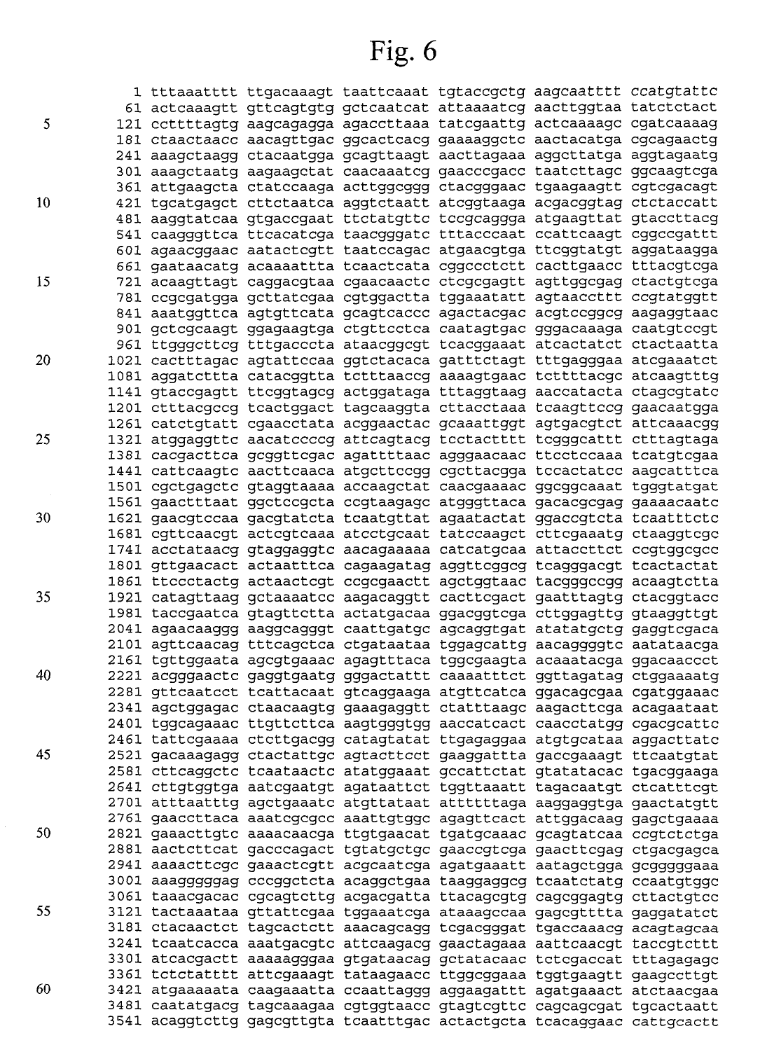

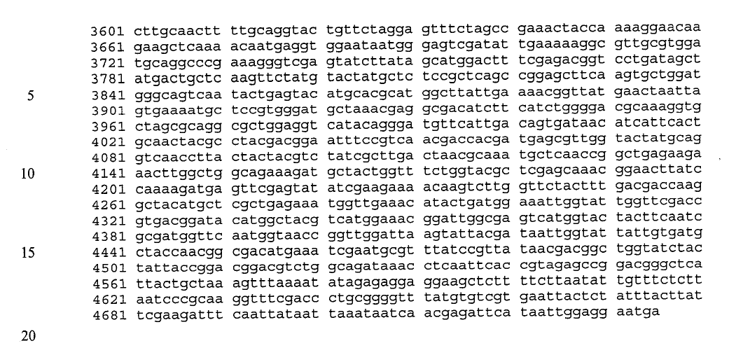

To study electron microscopy imaging, S. pneumoniae serogroup 14 was exposed to only 50 U/ml of Pal for 1 min. Specifically, S. pneumoniae strain DCC 1490 was grown in BHI to logarithmic phase, centrifuged and resuspended in sterile saline to an absorbance at 600 nm of 1.0. 500 ul of the suspension were incubated at room temperature with 500 ul of Pal at a final concentration of 50 U/ml. The lytic reaction was stopped after 10 sec, 1 min and 5 min by addition of glutaraldehyde (final concentration 2.5%). Bacteria and debris were pelleted by centrifugation and overlaid with 2.5% glutaraldehyde in 0.1 M cacodylate buffer (pH7.4). The samples were then postfixed in 1% osmium tetroxide, block stained with uranyl acetate and processed according to standard procedures by The Rockefeller University Electron Microscopy Service. Electron microscopy shown in FIG. 3 reveals protrusions of the cell membrane and the cytoplasm through single breaks in the cell wall, which appeared predominantly near the septum of the dividing diplococci (FIG. 3B). After 5 min, empty cell walls remained, retaining their original shape, indicating that digestion of amide bonds in a restricted location within the cell wall is sufficient for cell death (FIG. 3D). FIG. 3A shows the cell prior to exposure to the enzyme, and FIG. 3C shows the cell as it is dying.

The ability of Pal to eradicate S. pneumoniae from a mucosal surface was then tested in vivo in a mouse model of nasopharyngeal colonization following the model of H. Y. Wu, et al., Microb. Pathog. 23, 127-37 (1997), (incorporated by reference) with minor modifications. S. pneumoniae strain DCC 1490 was grown to logarithmic phase, centrifuged and resuspended to a predefined titer of 1010 cfu/ml. Swiss CD-I mice (weight range 22 to 24 g, Charles River Laboratories, Wilmington, MA) were anesthetized with a mixture of ketamine (Fort Dodge Animal Health, Fort Dodge, Iowa, 1.2 mg/animal) and xylazine (Miles Inc., Shawnee Mission, Kans., 0.25 mg/animal), and inoculated in one nostril with 10 ul of the bacterial suspension (n=18) or 10 ul of sterile saline (n=3). Forty-two hours later, inoculated animals were again anesthetized and 25 ul of Pal (350 U, n=9) or enzyme buffer (n=9) was instilled in each nostril over several minutes. The mouth of each animal was rinsed with additional 50 ul Pal (700 U), for a total of 1400 U. Five hours later, all animals were euthanized and the nasal cavity was washed through the dissected trachea with 60 ul of sterile saline. The nasal wash was serially 10-fold diluted and plated on blood agar for titer determination. The following day, alpha-hemolytic colonies were respread on blood agar and incubated with an optochin disk (BBL, Sparks, Md.). Bacteria with a zone of inhibition >14 mm were considered to be S. pneumoniae. Groups were compared with the Mann-Whitney test.

Treatment with Pal eliminated S. pneumoniae to undetectable levels (Log₁₀ 0 cfu/10 ul nasal wash) as opposed to treatment with buffer only (median [range] Log10 3.0 [2.0-3.0] cfu/10 ul, p<0.001) (FIG. 4A). The experiment was repeated with a lower dose of enzyme, randomizing the animals (n=16) for treatment with a total of 700 U of Pal or buffer. Enzyme treatment here completely eliminated pneumococci from 5 of 8 animals and significantly decreased titers in the remaining 3 (p<0.001) (FIG. 4B). Each experiment included 3 uncolonized control animals that revealed no S. pneumoniae. These results indicate that pneumococci on mucosal surfaces are highly susceptible to the action of the lytic enzyme.

To determine if repeated exposure to low concentrations of Pal enzyme is able to select for enzyme-resistant S. pneumoniae, strain DCC 1490 was grown on blood agar plates and exposed to low concentrations of Pal (<1 U). Colonies at the periphery of a clearing zone were picked, grown to logarithmic phase, streaked on a fresh plate and re-exposed to Pal. Sixteen rounds of exposure did not result in decrease of susceptibility to Pal when compared to the unexposed strain using the in vitro killing assay (p=NS (nonsignificant)), suggesting that resistance to Pal may occur at a very low frequency. It has been shown that the cell wall receptor for Pal as well as other pneumococcal phage lytic enzymes is choline, a molecule that is necessary for pneumococcal viability (R. Lopez, E. Garcia, P. Garcia, J. L. Garcia, Microb Drug Resist 3, 199-211. (1997), A. Tomasz, Science 157, 694-7. (1967), incorporated herein by reference). While not yet proven, it is possible that during a phage's association with bacteria over the millennia, to avoid being trapped inside the host, the binding domain of lytic enzymes has evolved to target a unique and essential molecule in the bacterial cell wall, making resistance to these enzymes a rare event.

Because the action of phage lytic enzymes is specific for a structure found in the bacterial peptidoglycan, and such structures are not present in mammalian tissues, it is anticipated that its effect on the human mucous membrane will be minimal or nonexistent. Also, no immune response is expected from nasal treatment with micrograms of Pal, since co-administration of higher protein concentrations with a mucosal adjuvant is generally necessary to elicit efficient mucosal immunity (L. Haan, et al., Vaccine 19, 2808-907 (2001).

It has been known for decades that the human upper respiratory mucosa is the reservoir for S. pneumoniae in the community. However, approaches to eliminate this reservoir have hitherto been of limited success because of the lack of specific reagents for this purpose. Through the use of phage lytic enzymes, nasopharyngeal colonization by S. pneumoniae can be controlled. It has been shown through these experiments that within seconds after contact, Pal is able to kill 15 clinical strains of S. pneumoniae, including the most frequently isolated serogroups and penicillin resistant strains, in vitro. Treatment of mice with Pal was also able to eliminate or significantly reduce nasal carriage of serotype 14 in a dose-dependent manner. Furthermore, because it has been found that the action of Pal, like other phage lytic enzymes, but unlike antibiotics, was rather specific for the target pathogen, it is likely that the normal flora will remain essentially intact (M. J. Loessner, G. Wendlinger, S. Scherer, Mol Microbiol 16, 1231-41. (1995) incorporated herein by reference).

While a Dp 1 phage was used to produce Pal which specifically kills Streptococcus pneumoniae, other phages may be used to produce an enzyme specific for Streptococcus pneumoniae.

These enzymes may be used alone or preferably in a variety of carriers to treat the illnesses caused by S. pneumoniae. For example, if there is a bacterial infection of the upper respiratory tract, the infection can be treated with a composition comprising an effective amount of at least one lytic enzyme specific for S. pneumoniae, and a carrier for delivering the lytic enzyme to a mouth, throat, or nasal passage. Preferably the enzyme would be Pal. The lytic enzyme may be produced by directly infecting Streptococcus pneumoniae with a phage specific for S. pneumoniae, and producing a lytic enzyme specific for S. pneumoniae. Alternatively, the lytic enzyme may be produced by the recombinant methods discussed, supra. If an individual has been exposed to someone with the upper respiratory infection, the lytic enzyme may be applied to mucosal lining to prevent any colonization of the infecting bacteria.

The composition which may be used for the prophylactic and therapeutic treatment of a S. pneumoniae infection includes the lytic enzyme and a means of application (such as a carrier system or an oral delivery mode) to the mucosal lining of the oral and nasal cavity, such that the enzyme is put in the carrier system or oral delivery mode to reach the mucosa lining.

Prior to, or at the time the lytic enzyme is put in the carrier system or oral delivery mode, it is preferred that the enzyme be in a stabilizing buffer environment for maintaining a pH range between about 4.0 and about 9.0, more preferably between about 5.5 and about 7.5.

The stabilizing buffer should allow for the optimum activity of the lysin enzyme. The buffer may contain a reducing reagent, such as dithiothreitol. The stabilizing buffer may also be or include a metal chelating reagent, such as ethylenediaminetetracetic acid disodium salt, or it may also contain a phosphate or citrate-phosphate buffer, or any other buffer.

Means of application of the lytic enzyme include, but are not limited to direct, indirect, carrier and special means or any combination of means. Direct application of the lytic enzyme may be by nasal sprays, nasal drops, nasal ointments, nasal washes, nasal injections, nasal packings, bronchial sprays and inhalers, or indirectly through use of throat lozenges, mouthwashes or gargles, or through the use of ointments applied to the nasal nares, or the face or any combination of these and similar methods of application. The forms in which the lytic enzyme may be administered include but are not limited to lozenges, troches, candies, injectants, chewing gums, tablets, powders, sprays, liquids, ointments, and aerosols.

When the lytic enzyme is introduced directly by use of nasal sprays, nasal drops, nasal ointments, nasal washes, nasal injections, nasal packing, bronchial sprays, oral sprays, and inhalers, the enzyme is preferably in a liquid or gel environment, with the liquid acting as the carrier. A dry anhydrous version of the enzyme may be administered by the inhaler and bronchial spray, although a liquid form of delivery is preferred.

The lozenge, tablet, or gum into which the lytic enzyme is added may contain sugar, corn syrup, a variety of dyes, non-sugar sweeteners, flavorings, any binders, or combinations thereof. Similarly, any gum based products may contain acacia, carnauba wax, citric acid, corn starch, food colorings, flavorings, non-sugar sweeteners, gelatin, glucose, glycerin, gum base, shellac, sodium saccharin, sugar, water, white wax, cellulose, other binders, and combinations thereof.

Lozenges may further contain sucrose, corn starch, acacia, gum tragacanth, anethole, linseed, oleoresin, mineral oil, and cellulose, other binders, and combinations thereof. In another embodiment of the invention, sugar substitutes are used in place of dextrose, sucrose, or other sugars.

As noted above, the enzyme may also be placed in a nasal spray, wherein the spray is the carrier. The nasal spray can be a long acting or timed release spray, and can be manufactured by means well known in the art. An inhalant may also be used, so that the phage enzyme may reach further down into the bronchial tract, including into the lungs.

Any of the carriers for the lytic enzyme may be manufactured by conventional means. However, it is preferred that any mouthwash or similar type products not contain alcohol to prevent denaturing of the enzyme. Similarly, when the lytic enzyme is being placed in a cough drop, gum, candy or lozenge during the manufacturing process, such placement should be made prior to the hardening of the lozenge or candy but after the cough drop or candy has cooled somewhat, to avoid heat denaturation of the enzyme.

The enzyme may be added to these substances in a liquid form or in a lyophilized state, whereupon it will be solubilized when it meets body fluids such as saliva. The enzyme may also be in a micelle or liposome.

The effective dosage rates or amounts of a lytic enzyme to treat the infection will depend in part on whether the lytic enzyme will be used therapeutically or prophylactically, the duration of exposure of the recipient to the infectious bacteria, the size and weight of the individual, etc. The duration for use of the composition containing the enzyme also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. The concentration of the active units of enzyme believed to provide for an effective amount or dosage of enzyme may be in the range of about 100 units/ml to about 500,000 units/ml of fluid in the wet or damp environment of the nasal and oral passages, and possibly in the range of about 100 units/ml to about 50,000 units/ml. More specifically, time exposure to the active enzyme units may influence the desired concentration of active enzyme units per ml. It should be noted that carriers that are classified as “long” or “slow” release carriers (such as, for example, certain nasal sprays or lozenges) could possess or provide a lower concentration of active (enzyme) units per ml, but over a longer period of time, whereas a “short” or “fast” release carrier (such as, for example, a gargle) could possess or provide a high concentration of active (enzyme) units per ml, but over a shorter period of time. The amount of active units per ml and the duration of time of exposure depends on the nature of infection, whether treatment is to be prophylactic or therapeutic, and other variables.

In another preferred embodiment, a mild surfactant in an amount effective to potentiate the therapeutic effect of the lytic enzyme may be used. Suitable mild surfactants include, inter alia, esters ofpolyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occurring surfactants, e.g., fatty acids, glycerides, monoglycerides, deoxycholate and esters of deoxycholate. While this treatment may be used in any mammalian species, the preferred use of this product is for a human.

As noted above, the phage enzymes, or their peptide fragments are directed to the mucosal lining, where, in residence, they kill colonizing disease bacteria. The mucosal lining, as disclosed and described herein, includes, for example, the upper and lower respiratory tract, eye, buccal cavity, nose, rectum, vagina, periodontal pocket, intestines and colon. Due to natural eliminating or cleansing mechanisms of mucosal tissues, conventional dosage forms are not retained at the application site for any significant length of time.

For these and other reasons it is advantageous to have materials which exhibit adhesion to mucosal tissues, to be administered with one or more phage enzymes and other complementary agents over a period of time. Materials having controlled release capability are particularly desirable, and the use of sustained release mucoadhesives has received a significant degree of attention.

J. R. Robinson (U.S. Pat. No. 4,615,697, incorporated herein by reference) provides a good review of the various controlled release polymeric compositions used in mucosal drug delivery. The patent describes a controlled release treatment composition which includes a bioadhesive and an effective amount of a treating agent. The bioadhesive is a water swellable, but water insoluble fibrous, crosslinked, carboxy functional polymer containing (a) a plurality of repeating units of which at least about 80 percent contain at least one carboxyl functionality, and (b) about 0.05 to about 1.5 percent crosslinking agent substantially free from polyalkenyl polyether. While the polymers ofRobinson are water swellable but insoluble, they are crosslinked, not thermoplastic, and are not as easy to formulate with active agents, and into the various dosage forms, as the copolymer systems of the present application. Micelles and multi lamillar micelles may also be used to control the release of enzyme.

Other approaches involving mucoadhesives which are the combination of hydrophilic and hydrophobic materials, are known. Orahesive.RTM. from E.R. Squibb & Co is an adhesive which is a combination of pectin, gelatin, and sodium carboxymethyl cellulose in a tacky hydrocarbon polymer, for adhering to the oral mucosa. However, such physical mixtures of hydrophilic and hydrophobic components eventually fall apart. In contrast, the hydrophilic and hydrophobic domains in the present invention produce an insoluble copolymer.

U.S. Pat. No. 4,948,580, also incorporated by reference, describes a bioadhesive oral drug delivery system. The composition, includes a freeze-driedpolymermixture formed of the copolymer poly(methyl vinyl ether/maleic anhydride) and gelatin, dispersed in an ointment base, such as mineral oil containing dispersed polyethylene. U.S. Pat. No. 5,413,792 (incorporated herein by reference) discloses paste-like preparations comprising (A) a paste-like base comprising a polyorganosiloxane and a water soluble polymeric material which are preferably present in a ratio by weight from 3:6 to 6:3, and (B) an active ingredient. U.S. Pat. No. 5,554,380 claims a solid or semisolid bioadherent orally ingestible drug delivery system containing a water-in-oil system having at least two phases. One phase comprises from about 25% to about 75% by volume of an internal hydrophilic phase and the other phase comprises from about 23% to about 75% by volume of an external hydrophobic phase, wherein the external hydrophobic phase is comprised of three components: (a) an emulsifier, (b) a glyceride ester, and (c)a wax material.

U.S. Pat. No. 5,942,243 describes some representative release materials useful for administering antibacterial agents according to embodiments of the invention.

An embodiment of the present invention features therapeutic compositions containing polymeric mucoadhesives consisting essentially of a graft copolymer comprising a hydrophilic main chain and hydrophobic graft chains for controlled release of biologically active agents. The graft copolymer is a reaction product of (1) a polystyrene macromonomer having an ethylenically unsaturated functional group, and (2) at least one hydrophilic acidic monomer having an ethylenically unsaturated functional group. The graft chains consist essentially of polystyrene, and the main polymer chain of hydrophilic monomeric moieties, some of which have acidic functionality. The weight percent of the polystyrene macromonomer in the graft copolymer is between about 1 and about 20% and the weight percent of the total hydrophilic monomer in the graft copolymer is between 80 and 99%, and wherein at least 10% of said total hydrophilic monomer is acidic, said graft copolymer when fully hydrated having an equilibrium water content of at least 90%.

Compositions containing the copolymers gradually hydrate by sorption of tissue fluids at the application site to yield a very soft jelly like mass exhibiting adhesion to the mucosal surface. During the period of time the composition is adhering to the mucosal surface it provides sustained release of the pharmacologically active agent, which is absorbed by the mucosal tissue.

Mucoadhesivity of the compositions of this invention is, to a large extent, produced by the hydrophilic acidic monomers of the chain in the polystyrene graft copolymer. The acidic monomers include, but are not limited to, acrylic and methacrylic acids, 2-acrylamido-2-methyl-propane sulfonic acid, 2-sulfoethyl methacrylate, and vinyl phosphonic acid. Other copolymerizable monomers include, but are not limited to N,N-dimethylacrylamide, glyceryl methacrylate, polyethylene glycol monomethacrylate, etc.

The compositions of the present invention may optionally contain other polymeric materials, such as poly(acrylic acid), poly,-(vinyl pyrrolidone), and sodium carboxymethyl cellulose plasticizers, and other pharmaceutically acceptable excipients in amounts that do not cause deleterious effect upon mucoadhesivity of the composition. The dosage forms of the compositions of this invention can be prepared by conventional methods.

In order to accelerate treatment of the infection, the therapeutic agent may further include at least one complementary agent which can also potentiate the bactericidal activity of the lytic enzyme. The complementary agent can be erythromycin, clarithromycin, azithromycin, roxithromycin, other members of the macrolide family, penicilins, cephalosporins, and any combinations thereof in amounts which are effective to synergistically enhance the therapeutic effect of the lytic enzyme. Virtually any other antibiotic may be used with the lytic enzyme. Similarly, other lytic enzymes may be included in the carrier to treat other bacterial infections.

Additionally, the therapeutic agent may further comprise the enzyme lysostaphin for the treatment of any Staphylococcus aureus bacteria present along with the S. pneumoniae. Mucolytic peptides, such as lysostaphin, have been suggested to be efficacious in the treatment of S. aureus infections of humans (Schaffner et al., Yale J. Biol. & Med., 39:230 (1967) and bovine mastitis caused by S. aureus (Sears et al., J. Dairy Science, 71 (Suppl. 1): 244(1988)). Lysostaphin, a gene product of Staphylococcus simulans, exerts a bacteriostatic and bactericidal effect upon S. aureus by enzymatically degrading the polyglycine crosslinks of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues. Furthermore, lysostaphin is also active against non-dividing cells, while most antibiotics require actively dividing cells to mediate their effects (Dixon et al., Yale J. Biology and Medicine, 41: 62-68 (1968)). Lysostaphin, in combination with the lytic enzyme, can be used in the presence or absence of the listed antibiotics. There is a degree of added importance in using both lysostaphin and the lytic enzyme in the same therapeutic agent. Frequently, when a body has a bacterial infection, the infection by one genus of bacteria weakens the body or changes the bacterial flora of the body, allowing other potentially pathogenic bacteria to infect the body. One of the bacteria that sometimes co-infects a body is Staphylococcus aureus. Many strains of Staphylococcus aureus produce penicillinase, such that Staphylococcus, Streptococcus, and other Gram positive bacterial strains will not be killed by standard antibiotics. Consequently, the use of the lytic and lysostaphin, possibly in combination with antibiotics, can serve as the most rapid and effective treatment of bacterial infections. In yet another preferred embodiment, the invention may include mutanolysin, and lysozyme.

It is also to be remembered that a carrier may have more than one lytic enzyme. For instance, a throat lozenge may comprise just a lytic enzyme or it may also include the lytic enzymes for, example, Haemophilus influenzae.

Similarly, lower respiratory illness (i.e. pneumoniae) may be treated with the lytic enzyme for Streptococcus pneumoniae. Similar methods and techniques may be used to treat pneumoniae as was used to treat upper respiratory illnesses. Treatment may be more dependent on the use of inhalers and any other device or carrier which will get the lytic enzymes into the lungs. Additionally, to more effectively treat the pneumoniae, the lytic enzyme should be given intravenously.

The method for treating systemic or tissue bacterial infections caused by Streptococcus pneumoniae comprises parenterally treating the infection with a therapeutic agent comprising an effective amount of at least one lytic enzyme specific for S. pneumoniae, and an appropriate carrier. A number of other different methods may be used to introduce the lytic enzyme(s). These methods include introducing the lytic enzyme intravenously, intramuscularly, subcutaneously, intrathecally, and subdermally. Intrathecal use would be most beneficial for treatment of bacterial meningitis.

In one preferred embodiment of the invention, infections may be treated by injecting into the infected tissue of the patient a therapeutic agent comprising the appropriate lytic enzyme(s) and a carrier for the enzyme. The carrier may be comprised of distilled water, a saline solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils, liposomes, and ethyl oleate are also useful herein.

In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. The pharmaceutical preparations according to the present invention are provided sterile and pyrogen free.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl.sub.2, CaCl.sub.2, etc.

Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), preferably 1.0 to 50% more preferably about 20%.

DMSO, is an aprotic solvent with a remarkable ability to enhance penetration of many locally applied drugs. DMSO may be diluted in sterile water for injection, or sodium chloride injection, or other pharmaceutically acceptable aqueous injection fluid, and used in concentrations of 0.1 to 1 00% (v/v).

The carrier vehicle may also include Ringer's solution, a buffered solution, and dextrose solution, particularly when an intravenous solution is prepared.

Prior to, or at the time the lytic enzyme is put in the carrier system or oral delivery mode, it is preferred that the enzyme be in a stabilizing buffer environment for maintaining a pH range between about 4.0 and about 9.0, more preferably between about 5.5 and about 7.5.

The stabilizing buffer should allow for the optimum activity of the lytic enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer. The buffers found in the carrier can serve to stabilize the environment for the lytic enzymes.

The effective dosage rates or amounts of the lytic enzyme to be administered parenterally, and the duration of treatment will depend in part on the seriousness of the infection, the weight of the patient, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 10,000,000 units/ml of composition, preferably in the range of about 1000 units/ml to about 10,000,000 units/ml, and most preferably from about 10,000 to 10,000,000 units/ml. The amount of active units per ml and the duration of time of exposure depends on the nature of infection, and the amount of contact the carrier allows the lytic enzyme to have. It is to be remembered that the enzyme works best when in a fluid environment. Hence, effectiveness of the enzyme is in part related to the amount of moisture trapped by the carrier. For the treatment of a septicemic infection, for pneumoniae, or bacterial meningitis, there should be a continuous intravenous flow of therapeutic agent into the blood stream. The concentration of lytic enzyme for the treatment of septicemia is dependent upon the bacterial count in the blood and the blood volume.

In order to accelerate treatment of the infection, the therapeutic agent may further include at least one complementary agent which can also potentiate the bactericidal activity of the lytic enzyme. The complementary agent can be any antibiotic effective against Streptococcus pneumoniae. Similarly, other lytic enzymes may be included to treat other bacterial infections.

Additionally, the therapeutic agent may further comprise the enzyme lysostaphin, a lytic enzyme for the treatment of any Staphylococcus aureus bacteria. In yet another preferred embodiment, the invention may include mutanolysin, and lysozyme Another use of the invention is for the prophylactic and therapeutic treatment of eye infections, such as conjunctivitis. The method of treatment comprises administering eye drops or an eye wash which comprise an effective amount of at least one lytic enzyme genetically coded for by a bacteriophage specific for Streptococcus pneumoniae and a carrier capable of being safely applied to an eye, with the carrier containing the lytic enzyme. The eye drops or eye wash are preferably in the form of an isotonic solution. The pH of the solution should be adjusted so that there is no irritation of the eye, which in turn would lead to possible infection by other organisms, and possible to damage to the eye. While the pH range should be in the same range as for other lytic enzymes, the most optimal pH will be in the range of from 6.0 to 7.5. Similarly, buffers of the sort described above for the other lytic enzymes should also be used. Other antibiotics which are suitable for use in eye drops may be added to the composition containing the lytic enzymes. Bactericides and bacteriostatic compounds may also be added. The concentration of the enzyme in the solution can be in the range of from about 100 units/ml to about 500,000 units/ml, with a more preferred range of about 100 to about 5,000 units/ml, and about 100 to about 50,000 units/ml.

The lytic enzyme described above may also be used in a contact lens solution, for the soaking and cleaning of contact lenses. This solution, which is normally an isotonic solution, may contain, in addition to the enzyme, sodium chloride, mannitol and other sugar alcohols, borates, preservatives, etc.

This lytic enzyme may also be used to treat ear infections caused by Streptococcus pneumoniae. Otitis media is an inflammation of the middle ear characterized by symptoms such as otaigia, hearing loss and fever. One of the primary causes of these symptoms is a build up of fluid (effusion) in the middle ear. Complications include permanent hearing loss, perforation of the tympanic membrane, acquired cholesteatoma, mastoiditis, and adhesive otitis. Children who develop otitis media in the first years of life are at risk for recurrent acute or chronic disease.

One of the primary causes of otitis media is Streptococcus pneumoniae. It is thought that S. pneumoniae causes otitis media by adhering to nasopharyngeal cells. The adherence of S. pneumoniae to nasopharyngeal cells causes those cells to become infected and to produce secretions. The middle ear becomes infected because mechanical or functional obstruction of the Eustachian tube, which protects the middle ear from nasopharyngeal secretions, results in negative middle ear pressure. This negative pressure causes the nasopharyngeal secretions to enter the middle ear resulting in an infection, such as otitis media, usually with effusion.

The lytic enzyme (genetically coded for by a bacteriophage specific for Streptococcus neumoniae, wherein the lytic enzyme specifically lyses the cell wall of said Streptococcus neumoniae) may be applied to an infected ear by delivering the enzyme in an appropriate carrier to the canal of the ear. The carrier may comprise sterile aqueous or oily solutions or suspensions. The lytic enzyme may be added to the carrier, which may also contain suitable preservatives, and preferably a surface active agent. Bactericidal and fungicidal agents preferably included in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol. Additionally, any number of other ear drop carriers may be used.

The concentrations and preservatives used for the treatment of otitis media and other similar ear infections are the same as discussed for eye infections, and the carrier into which the enzyme goes is similar or identical to the carriers for treatment of eye infections. Additionally, the carrier may typically includes vitamins, minerals, carbohydrates, sugars, amino acids, proteinaceous materials, fatty acids, phospholipids, antioxidants, phenolic compounds, isotonic solutions, oil based solutions, oil based suspensions, and combinations thereof.

Endocarditis is commonly caused by Streptococcal infections, including Streptococcus pneumoniae. Streptococcus pneumoniae, as well as certain other Streptococcal species, may grow in the heart valves of an infected patient and cause damage thereto. Endocarditis is currently diagnosed by clinical features, echo cardiogram, the presence of heart murmurs, and positive blood cultures. Patients with rheumatic fever, damaged heart valves or prosthetic valves are at risk of a secondary streptococcal infection leading to endocarditis when having routine dental or gastrointestinal procedures.

Current therapy for endocarditis involves long term IV antibiotics; however, some of the antibiotics necessary to treat endocarditis are potentially toxic, such as vancomycin and gentamicin which may be nephrotoxic and ototoxic.

As an alternative or supplement to the use of antibiotics for endocarditis, a lytic enzyme may be used for the treatment of endocarditis. The enzyme may be preferablly administered parenterally, and, perhaps under certain conditions, intramuscularly, subcutaneously, and subdermally. The carrier may be comprised of distilled water, a saline solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils, liposomes, and ethyl oleate are also useful herein.

In cases where perenteral injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. The pharmaceutical preparations according to the present invention are provided sterile and pyrogen free.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl.sub.2, CaCl.sub.2, etc.

Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of from about 0.1 to 100% (v/v), preferably about 1.0 to about 50% more preferably about 20%.

DMSO is an aprotic solvent with a remarkable ability to enhance penetration of many locally applied drugs. DMSO may be diluted in sterile water for injection, or sodium chloride injection, or other pharmaceutically acceptable aqueous injection fluid, and used in concentrations of from about 0.1 to 100% (v/v).

The carrier vehicle may also include Ringer's solution, a buffered solution, and dextrose solution, particularly when an intravenous solution is prepared.

Prior to, or at the time the lytic enzyme is put in the carrier system or oral delivery mode, it is preferred that the enzyme be in a stabilizing buffer environment for maintaining a pH range between about 4.0 and about 9.0, more preferably between about 5.5 and about 7.5.

The stabilizing buffer should allow for the optimum activity of the lytic enzyme. The buffer may be contain reducing reagent, such as dithiothreitol. The stabilizing buffer may also be or include a metal chelating reagent, such as ethylenediaminetetracetic acid disodium salt, or it may also contain a phosphate or citrate-phosphate buffer. The buffers found in the carrier can serve to stabilize the environment for the lytic enzymes.

The effective dosage rates or amounts of the lytic enzyme to be administered parenterally, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 500,000 units/ml of composition, preferably in the range of about 1000 units/ml to about 5,000,000 units/ml, and most preferably from about 10,000 to 5,000,000 units/ml. The amount of active units per ml and the duration of time of exposure depends on the nature of infection, and the amount of contact the carrier allows the lytic enzyme to have.

Many modifications and variations of the present invention are possible in light of the above teachings. Such other modifications and variations which will be readily apparent to a skilled are included within the spirit and scope of the attached claims.

What we claim is:

1) A method for the prophylactic or therapeutic treatment of Streptococcus pneumoniae, comprising:
administering to the site of an infection or colonization an effective amount of at least one lytic enzyme genetically coded for by a bacteriophage specific for Streptococcus pneumoniae, wherein said at least one lytic enzyme specifically lyses the cell wall of said Streptococcus pneumoniae.

2) The method according to claim 1, wherein said at least one lytic enzyme is produced by infecting said Streptococcus pneumoniae with a bacteriophage specific for said Streptococcus pneumoniae.

3) The method according to claim 1, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

4) The method according to claim 1, wherein said at least one lytic enzyme is produced by removing a gene for the lytic enzyme from the phage genome, introducing said gene into a transfer vector, and cloning said transfer vector into an expression system.

5) The method according to claim 4, wherein said transfer vector is a plasmid.

6) The method according to claim 4, wherein said expression system is a bacteria.

7) The method according to claim 6, wherein said bacteria is selected from the group consisting of E. coli and Bacillus.

8) The method according to claim 4, wherein said expression system is a cell free expression system.

9) The method according to claim 1, further comprising delivering said lytic enzyme in a carrier suitable for delivering said lytic enzyme to the site of the infection.

10) The method according to claim 9, wherein said carrier is selected from the group consisting of nasal sprays, nasal drops, nasal inhalants, nasal ointments, nasal washes, nasal injections, nasal drops, nasal ointments, nasal washes, nasal injections, gels, nasal packings, ointments, lozenges, troches, candies, injectants, chewing gums, tablets, powders, sprays, injectants, powders, and liquids.

11) The method according to claim 8, further comprising delivering a dry anhydrous version of the enzyme by an inhaler.

12) The method according to claim 1, further comprising delivering said lytic enzyme parenterally.

13) The method according to claim 12, wherein said lytic enzyme is delivered intravenously.

14) The method according to claim 12, wherein said lytic enzyme is delivered intramuscularly.

15) The method according to claim 12, wherein said lytic enzyme is delivered subdermally.

16) The method according to claim 12, wherein said lytic enzyme is delivered intrathecally.

17) The method according to claim 1, wherein said bacteriophage is selected from the group consisting of Dp-1, Dp-4, Cp-1, Cp-7, Cp-9, Cp-5, MM1, EJ-1,HB-3, HB-623, HB-746, ω-1, and ω-2.

18) The method according to claim 17, wherein said bacteriophage is Dp-1.

19) The method according to claim 1, further comprising an antibiotic.

20) A method for treating an upper respiratory tract illness or colonization caused by
Streptococcus pneumoniae, comprising administering to a mouth, throat, or nasal passage of a mammal a composition comprising an effective amount of at least one lytic enzyme genetically coded for by a bacteriophage specific for Streptococcus pneumoniae, wherein said at least one lytic enzyme the cell wall of said Streptococcus pneumoniae.

21) The method according to claim 20, wherein said composition further comprises a carrier selected from the group consisting of nasal sprays, nasal drops, nasal inhalants, nasal ointments, nasal washes, nasal injections, nasal drops, nasal ointments, nasal washes, nasal injections, gels, nasal packings, ointments, lozenges, troches, candies, injectants, chewing gums, tablets, powders, sprays, injectants, powders, intravenous solution, and liquids.

22) The method according to claim 20, wherein said composition further comprises a buffer that maintains pH of the composition at a range between about 4.0 and about 9.0.

23) The method according to claim 22, wherein said buffer comprises a reducing reagent.

24) The method according to claim 23, wherein said reducing reagent is dithiothreitol.

25) The method according to claim 22, wherein said buffer comprises a metal chelating reagent.

26) The method according to claim 25, wherein said metal chelating reagent is ethylenediaminetetracetic disodium salt.

27) The method according to claim 22, wherein said buffer is a citrate-phosphate buffer.

28) The method according to claim 22, further comprising a bactericidal or bacteriostatic agent as a preservative.

29) The method according to claim 20, wherein said lytic enzyme is lyophilized.

30). The method according claim 20, wherein said at least one lytic enzyme is present in a concentration of about 100 to about 500,000 active enzyme units per milliliter of fluid in the wet environment of the nasal or oral passages.

31) The method according to claim 20, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

32) The method according to claim 20, wherein said composition is administered parenterally.

33) The method according to claim 20, wherein said lytic enzyme is produced by infecting said Streptococcus pneumoniae with a bacteriophage specific for said Streptococcus pneumoniae.

34) The method according to claim 20, wherein said at least one lytic enzyme is produced by removing a gene for the lytic enzyme from the phage genome, putting said gene into a transfer vector, and cloning said transfer vector into an expression system.

35) The method according to claim 34, wherein said transfer vector is a plasmid.

36) The method according to claim 34, wherein said expression system is a bacteria.

37) The method according to claim 36, wherein said bacteria is selected from the group consisting essentially of E. coli and Bacillus.

38) The method according to claim 34, wherein said expression system is a cell free expression system.

39) A method of treating bacterial meningitis caused by Streptococcus pneumoniae, comprising administering a composition comprising an effective amount of at least one lytic enzyme genetically coded for by a bacteriophage specific for Streptococcus pneumoniae, wherein said at least one lytic enzyme specifically lyses the cell wall of said Streptococcus pneumoniae.

40) The method according to claim 39, wherein said composition is administered parenterally.

41) The method according to claim 39, wherein said composition is administered intravenously.

42) The method according to claim 39, wherein said composition is administered subcutaneously.

43) The method according to claim 39, wherein said composition is administered intramuscularly.

44) The method according to claim 39, wherein said composition is administered intrathecally.

45) The method according to claim 39, wherein said at least one lytic enzyme is produced by infecting said Streptococcus pneumoniae with a bacteriophage specific for said Streptococcus pneumoniae.

46) The method according to claim 39, wherein said at least one lytic enzyme is produced by removing a gene for the lytic enzyme from the phage genome, putting said gene into a transfer vector, and cloning said transfer vector into an expression system.

47) The method according to claim 46, wherein said transfer vector is a plasmid.

48) The method according to claim 46, wherein said expression system is a bacteria.

46) The method according to claim 48, wherein said bacteria is selected from the group consisting of E. coli and Bacillus. .

47) The method according to claim 43, wherein said expression system is a cell free expression system.

48) The method according to claim 39, wherein said composition further comprises a carrier.

49) The method according to claim 48, wherein said carrier is selected from the group consisting of distilled water, a saline solution, albumin, a serum, Ringer's solution, a buffered solution, a dextrose solution, and combinations thereof.

50) The method according to claim 48, wherein said carrier comprises additives selected from the group consisting of p-hydroxybenzoates, stabilizers, fixed oils, ethyl oleate, neutral salts, dextrose, trehalose, dextrins, lactose, phosphate buffered saline, gelatin, albumin, vasoconstriction agents, organic acids organic acid salts, antioxidants, low molecular weight polypeptides, proteins, immunoglobulins, hydrophilic polymers, amino acids, monosaccharides, disaccharides, other carbohydrates including cellulose or its derivatives, glucose, chelating agents, sugar alcohols, counter-ions, non-ionic surfactants, glycerin, glycerol, DMSO, and combinations thereof.

51) A method for treating, preventing or ameliorating a Streptococcus pneumoniae infection at a mucosal surface, comprising the steps of:
a) obtaining a composition comprising an effective amount of at least one lytic enzyme genetically coded for by a bacteriophage specific for Streptococcus pneumoniae, wherein said at least one lytic enzyme specifically lyses the cell wall of said Streptococcus pneumoniae, and
b) applying said composition to the mucosal surface.

52) The method according to claim 51, wherein said bacteriophage is selected from the group consisting of Dp-1, Dp-4, Cp-1, Cp-7, Cp-9, Cp-5, MM1, EJ-1,HB-3, HB-623, HB-746, ω-1, and ω-2.

53) A method of treating illnesses or infections of Streptococcus pneumoniae, comprising administering parenterally a composition comprising an effective amount of at least one lytic enzyme genetically coded for by a bacteriophage specific for Streptococcus pneumoniae, wherein said at least one lytic enzyme specifically lyses the cell wall of said Streptococcus pneumoniae without affecting any other bacterial flora present.

54) The method according to claim 53, wherein said at least one lytic enzyme is produced by infecting said Streptococcus pneumoniae with a bacteriophage specific for said Streptococcus pneumoniae.

55) The method according to claim 53, wherein said at least one lytic enzyme is produced by removing a gene for the lytic enzyme from the phage genome, putting said gene into a transfer vector, and cloning said transfer vector into an expression system.

56) The method according to claim 55, wherein said transfer vector is a plasmid.

57) The method according to claim 55, wherein said expression system is a bacteria.

58) The method according to claim 57, wherein said bacteria is selected from the group consisting of E. coli and Bacillus.

59) The method according to claim 55, wherein said expression system is a cell free expression system

60) The method according to claim 53, further comprising delivering said lytic enzyme in a carrier suitable for delivering said lytic enzyme to the site of the infection.

61) A method for the treatment of a bacterial infection caused by Streptococcus pneumoniae, comprising the steps of:
(a) obtaining a composition comprising an effective amount of a lytic enzyme, said composition prepared by the steps of:
1) obtaining at least one lytic enzyme genetically coded for by a bacteriophage specific for Streptococcus pneumoniae, wherein said at least one lytic enzyme has the ability to specifically lyse the cell wall of said Streptococcus pneumoniae;
2) admixing said at least one lytic enzyme to a carrier suitable for delivery of said at least one lytic enzyme to the site of the infection; and
(b) administering said composition.

62) The method according to claim 61, wherein said at least one lytic enzyme is produced by infecting said Streptococcus pneumoniae with a bacteriophage specific for said Streptococcus pneumoniae.

63) The method according to claim 61, wherein said at least one lytic enzyme is produced by removing a gene for the lytic enzyme from the phage genome, putting said gene into a transfer vector, and cloning said transfer vector into an expression system.

64) The method according to claim 63, wherein said transfer vector is a plasmid.

65) The method according to claim 63, wherein said expression system is a bacteria.

66) The method according to claim 65, wherein said bacteria is selected from the group consisting of E. coli and Bacillus.

67) The method according to claim 63, wherein said expression system is a cell free expression system.

68) The method according to claim 61, further comprising delivering said lytic enzyme in a carrier suitable for delivering said lytic enzyme to the site of the infection.

69) The method according to claim 68, wherein said carrier is selected from the group consisting of nasal sprays, nasal drops, nasal inhalants, nasal ointments, nasal washes, nasal injections, nasal drops, nasal ointments, nasal washes, nasal injections, gels, nasal packings, ointments, lozenges, ttroches, candies, injectants, chewing gums, tablets, powders, sprays, injectants, powders, and liquids.

70) The method according to claim 61, further comprising delivering a dry anhydrous version of the enzyme by an inhaler.

71) The method according to claim 61, wherein said lytic enzyme is delivered parenterally.

72) The method according to claim 71, wherein said lytic enzyme is delivered intravenously.

73) The method according to claim 71, wherein said lytic enzyme is delivered intramuscularly.

74) The method according to claim 71, wherein said lytic enzyme is delivered subdermally.

75) The method according to claim 71, wherein said lytic enzyme is delivered intrathecally.

76) The method according to claim 61, wherein said bacteriophage is selected from the group consisting of Dp-1, DP-4, Cp-1, Cp-7, Cp-9, Cp-5, MM1, EJ-1,HB-3, HB-623, HB-746, ω-1, and ω-2.

77) The method according to claim 61, wherein said bacteriophage is Dp-1.

78) The method according to claim 61, further comprising an antibiotic.

79) The method according to claim 61, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

80) A method of treating eyes exposed to Streptococcus pneumoniae, comprising:
administering to the eyes an effective amount of at least one lytic enzyme genetically coded for by a bacteriophage specific for Streptococcus pneumoniae, wherein said at least one lytic enzyme specifically lyses the cell wall of said Streptococcus pneumoniae.

81) The method according to claim 80, wherein said at least one lytic enzyme is produced by infecting said Streptococcus pneumoniae with a bacteriophage specific for said Streptococcus pneumoniae.

82) The method according to claim 80, wherein said at least one lytic enzyme is produced by removing a gene for the lytic enzyme from the phage genome, putting said gene into a transfer vector, and cloning said transfer vector into an expression system.

83) The method according to claim 82, wherein said transfer vector is a plasmid.

84) The method according to claim 81, wherein said expression system is a bacteria.

85) The method according to claim 84, wherein said bacteria is selected from the group consisting of E. coli and Bacillus. .

86) The method according to claim 81, wherein said expression system is a cell free expression system

87) The method according to claim 80, further comprising delivering said lytic enzyme in a carrier suitable for delivering said lytic enzyme to the site of the infection.

88) The method according to claim 87, wherein said carrier is an eye drop solution.

89) The method according to claim 80, further comprising delivering a dry anhydrous version of the enzyme by an inhaler.

90) The method according to claim 80, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

91) The method according to claim 80, further comprising delivering said lytic enzyme parenterally.

92) The method according to claim 91, wherein said lytic enzyme is delivered intravenously.

93) The method according to claim 91, wherein said lytic enzyme is delivered intramuscularly.

94) The method according to claim 91, wherein said lytic enzyme is delivered subdermally.

95) The method according to claim 80, wherein said bacteriophage is selected from the group consisting of Dp-1, DP-4, Cp-1, Cp-7, Cp-9, Cp-5, MM1, EJ-1,HB-3, HB-623, HB-746, ω-1, and ω-2.

96) The method according to claim 95, wherein said bacteriophage is Dp-1.

97) A method for treating ear infections, comprising administering to a canal of an ear an effective amount of at least one lytic enzyme genetically coded for by a bacteriophage specific for Streptococcus pneumoniae, wherein said at least one lytic enzyme specifically lyses the cell wall of said Streptococcus pneumoniae.

98) The method according to claim 97, wherein said at least one lytic enzyme is produced by infecting said Streptococcus pneumoniae with a bacteriophage specific for said Streptococcus pneumoniae.

99) The method according to claim 97, wherein said at least one lytic enzyme is produced by removing a gene for the lytic enzyme from the phage genome, putting said gene into a transfer vector, and cloning said transfer vector into an expression system.

100) The method according to claim 99, wherein said transfer vector is a plasmid.

101) The method according to claim 99, wherein said expression system is a bacteria.

102) The method according to claim 97, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No.2 under stringent hybridization conditions.

103) The method according to claim 99, wherein said expression system is a cell free expression system

104) The method according to claim 97, further comprising delivering said lytic enzyme in a carrier suitable for delivering said lytic enzyme to the site of the infection.

105) The method according to claim 97, wherein said bacteriophage is selected from the group consisting of Dp-1, DP-4, Cp-1, Cp-7, Cp-9, Cp-5, MM1, EJ-1,HB-3, HB-623, HB-746, ω-1, and ω-2.

106) The method according to claim 105, wherein said bacteriophage is Dp-1.

107) The method according to claim 97, further comprising an antibiotic.

108) The method according to claim 104, wherein said carrier is selected from the group consisting of vitamins, minerals, carbohydrates, sugars, amino acids, proteinacious materials, fatty acids, phospholipids, antioxidants, phenolic compounds, isotonic solutions, oil based solutions, oil based suspensions, and combinations thereof.

109) A method for preventing infection of contact lens solution by Streptococcus pneumoniae, comprising the steps of:
administering to said contact lens solution an effective amount of at least one lytic enzyme genetically coded for by a bacteriophage specific for said Streptococcus pneumoniae, wherein said at least one lytic enzyme specifically lyses the cell wall of said Streptococcus pneumoniae.

110) The method according to claim 109, wherein said at least one lytic enzyme is produced by infecting said Streptococcus pneumoniae with a bacteriophage specific for said Streptococcus pneumoniae.

111) The method according to claim 109, wherein said at least one lytic enzyme is produced by removing a gene for the lytic enzyme from the phage genome, putting said gene into a transfer vector, and cloning said transfer vector into an expression system.

112) The method according to claim 111, wherein said transfer vector is a plasmid.

113) The method according to claim 111, wherein said expression system is a bacteria.

114) The method according to claim 111, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

115) The method according to claim 111, wherein said expression system is a cell free expression system

116) The method according to claim 109, further comprising delivering said lytic enzyme in a carrier suitable for delivering said lytic enzyme to the site of the infection.

117) The method according to claim 109, wherein said bacteriophage is selected from the group consisting of Dp-1, DP-4, Cp-1, Cp-7, Cp-9, Cp-5, MM1, EJ-1,HB-3, HB-623, HB-746, ω-1, and ω-2.

118) The method according to claim 117, wherein said bacteriophage is Dp-1.

119) The method according to claim 109, wherein said contact lens solution further comprising an antibiotic.

120) The method according to claim 109, wherein said contact lens solution is an isotonic solution.

121) The method according to claim 109, wherein said contact lens solution further comprises sodium chloride, sugar alcohols, borates, preservatives, and combinations thereof.

122) A method for treating endocarditis caused by Streptococcus pneumoniae, comprising administering to site of the infection an effective amount of at least one lytic enzyme genetically coded for by a bacteriophage specific for said Streptococcus pneumoniae, wherein said at least one lytic enzyme specifically lyses the cell wall of said Streptococcus pneumoniae.

123) The method according to claim 122, wherein said at least one lytic enzyme is produced by infecting said Streptococcus pneumoniae with a bacteriophage specific for said Streptococcus pneumoniae.

124) The method according to claim 122, wherein said at least one lytic enzyme is produced by removing a gene for the lytic enzyme from the phage genome, putting said gene into a transfer vector, and cloning said transfer vector into an expression system.

125) The method according to claim 124, wherein said transfer vector is a plasmid.

126) The method according to claim 124, wherein said expression system is a bacteria.

127) The method according to claim 122, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

128) The method according to claim 124, wherein said expression system is a cell free expression system

129) The method according to claim 122, further comprising delivering said lytic enzyme in a carrier suitable for delivering said lytic enzyme to the site of the infection.

130) The method according to claim 122, wherein said bacteriophage is selected from the group consisting of Dp-1, DP-4, Cp-1, Cp-7, Cp-9, Cp-5, MM1, EJ-1,HB-3, HB-623, HB-746, ω-1, and ω-2.

131) The method according to claim 130, wherein said bacteriophage is Dp-1.

132) The method according to claim 129, wherein said carrier is selected from the group consisting of distilled water, a saline solution, albumin, a serum, fixed oils, liposomes, ethyl oleate, and combinations thereof.

133) The method according to claim 132, wherein said carrier may further comprise preservatives, stabilizers, buffers, gelatin, a vasoconstriction agent, amino acids, antioxidants, polypeptides, hydrophilic polymers, sugar alcohols, chelating agents, sugars, counter ions, non-ionic surfactants, and combinations thereof.

134) The method according to claim 129, wherein said composition is administered parenterally.

135) The method according to claim 134, wherein said composition is administered intramuscularly.

136) The method according to claim 134, wherein said composition is administered intravenously.

137) The method according to claim 134, wherein said composition is administered subdermally.

138) A method for the prophylactic treatment of Streptococcus pneumoniae, comprising
administering to the site of carriage an effective amount of at least one lytic enzyme genetically coded for by a bacteriophage specific for Streptococcus pneumoniae, wherein said at least one lytic enzyme specifically lyses the cell wall of said Streptococcus pneumoniae.

139) The method according to claim 138, wherein said at least one lytic enzyme is produced by infecting said Streptococcus pneumoniae with a bacteriophage specific for said Streptococcus pneumoniae.

140) The method according to claim 138, wherein said at least one lytic enzyme is produced by removing a gene for the lytic enzyme from the phage genome, introducing said gene into a transfer vector, and cloning said transfer vector into an expression system.

141) The method according to claim 140, wherein said transfer vector is a plasmid.

142) The method according to claim 140, wherein said expression system is a bacteria.

143) The method according to claim 142, wherein said bacteria is selected from the group consisting of wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

144) The method according to claim 140, wherein said expression system is a cell free expression system.

145) The method according to claim 138, further comprising delivering said lytic enzyme in a carrrier suitable for delivering said lytic enzyme to the site of the carriage.

146) The method according to claim 138, wherein said carrier is selected from the group consisting of nasal sprays, nasal drops, nasal inhalants, nasal ointments, nasal washes, nasal injections, nasal drops, nasal ointments, nasal washes, nasal injections, gels, nasal packings, ointments, lozenges, troches, candies, injectants, chewing gums, tablets, powders, sprays, injectants, powders, and liquids.

147) The method according to claim 138, further comprising delivering a dry anhydrous version of the enzyme by an inhaler.

148) The method according to claim 138, further comprising delivering a dry anhydrous version of the enzyme by a bronchial spray.

149) The method according to claim 138, further comprising delivering said lytic enzyme parenterally.

150) The method according to claim 138, wherein said lytic enzyme is delivered intravenously.

151) The method according to claim 12, wherein said lytic enzyme is delivered intramuscularly.

152) The method according to claim 12, wherein said lytic enzyme is delivered subdermally.

153) The method according to claim 1, wherein said bacteriophage is selected from the group consisting of Dp-1, Dp-4, Cp-1, Cp-7, Cp-9, Cp-5, MM1, EJ-1,HB-3, HB-623, HB-746, ω-1, and ω-2.

154) The method according to claim 153, wherein said bacteriophage is Dp-1.

155) A method for the treating the carriage of Streptococcus pneumoniae in the upper respiratory tract illness, comprising administering to a mouth, throat, or nasal passage of a mammal a composition comprising an effective amount of at least one lytic enzyme genetically coded for by a bacteriophage specific for Streptococcus pneumoniae, wherein said at least one lytic enzyme the cell wall of said Streptococcus pneumoniae.

156) The method according to claim 155, wherein said composition further comprises a carrier selected from the group consisting of nasal sprays, nasal drops, nasal inhalants, nasal ointments, nasal washes, nasal injections, nasal ointments, nasal injections, gels, nasal packings, ointments, lozenges, troches, candies, injectants, chewing gums, tablets, powders, sprays, injectants, powders, intravenous solution, and liquids.

157) The method according to claim 156, wherein said composition further comprises a buffer that maintains pH of the composition at a range between about 4.0 and about 9.0.

158) The method according to claim 157, wherein said buffer comprises a reducing reagent.

159) The method according to claim 158, wherein said reducing reagent is dithiothreitol.

160) The method according to claim 157, wherein said buffer comprises a metal chelating reagent.

161) The method according to claim 160, wherein said metal chelating reagent is ethylenediaminetetracetic disodium salt.

162) The method according to claim 157, wherein said buffer is a citrate-phosphate buffer.

163) The method according to claim 155, further comprising a bactericidal or bacteriostatic agent as a preservative.

164) The method according to claim 155, wherein said lytic enzyme is lyophilized.

165). The method according claim 155, wherein said at least one lytic enzyme is present in a concentration of about 100 to about 500,000 active enzyme units per milliliter of fluid in the wet environment of the nasal or oral passages.

166) The method according to claim 165, wherein said at least one lytic enzyme is present in a concentration of about 100 to about 50,000 active enzyme units per milliliter of fluid in the wet environment of the nasal or oral passages.

167) The method according to claim 155, wherein said composition is administered parenterally.

168) The method according to claim 155, wherein said lytic enzyme is produced by infecting said Streptococcus pneumoniae with a bacteriophage specific for said Streptococcus pneumoniae.

169) The method according to claim 155, wherein said at least one lytic enzyme is produced by removing a gene for the lytic enzyme from the phage genome, putting said gene into a transfer vector, and cloning said transfer vector into an expression system.

170) The method according to claim 169, wherein said transfer vector is a plasmid.

171) The method according to claim 169, wherein said expression system is a bacteria.

172) The method according to claim 171, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

173) The method according to claim 169, wherein said expression system is a cell free expression system

174) A composition for treating Streptococcus pneumoniae, comprising a lytic enzyme and a carrier, wherein the lytic enzyme is genetically coded by a bacteriophage specific for the Streptococcus pneumoniae, and wherein the enzyme is prepared by recombination.

175) The composition according to claim 174, wherein said transfer vector is a plasmid.

176) The composition according to claim 174, wherein said expression system is a bacteria.

177) The composition according to claim 176, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

178) The composition according to claim 176, wherein said expression system is a cell free expression system.

179) The composition according to claim 174, wherein said carrier is selected from the group consisting of nasal sprays, nasal drops, nasal inhalants, nasal ointments, nasal washes, nasal injections, nasal drops, nasal ointments, nasal washes, nasal injections, gels, nasal packings, lozenges, troches, candies, injectants, chewing gums, tablets, powders, sprays, injectants, powders, and liquids.

180) The composition according to claim 174, further comprising delivering a dry anhydrous version of the enzyme by an inhaler.

181) The composition according to claim 174, further comprising delivering a dry anhydrous version of the enzyme by a bronchial spray.

182) The composition according to claim 174, wherein said carrier is suitable for delivering the lytic enzyme parenterally.

183) The composition according to claim 174, wherein said carrier is suitable for delivering the lytic enzyme intravenously.

184) The composition according to claim 174, wherein said carrier is suitable for delivering said lytic enzyme intramuscularly.

185) The composition according to claim 174, wherein said carrier is suitable for delivering said lytic enzyme subdermally.

186) The composition according to claim 174, wherein said carrier is suitable for delivering said lytic enzyme intrathecally.

187) The composition according to claim 174, wherein said bacteriophage is selected from the group consisting of Dp-1, Dp-4, Cp-1, Cp-7, Cp-9, Cp-5, MM1, EJ-1,HB-3, HB-623, HB-746, ω-1, and ω-1.

188) The composition according to claim 187, wherein said bacteriophage is Dp-1.

189) The composition according to claim 174, further comprising an antibiotic.

190) A composition for treating an respiratory tract illnesses caused by Streptococcus pneumoniae, wherein said composition is formed by:
i) obtaining an effective amount of at least one lytic enzyme genetically coded for by a bacteriophage specific for Streptococcus pneumoniae, wherein said at least one lytic enzyme lyses the cell wall of said Streptococcus pneumoniae; and
ii) incorporating said lytic enzyme into a carrier which can deliver said lytic enzyme to said mouth, throat, or nasal passage of a mammal.

191) The composition according to claim 190, wherein said composition further comprises a carrier selected from the group consisting of nasal sprays, nasal drops, nasal inhalants, nasal ointments, nasal washes, nasal injections, nasal ointments, nasal injections, gels, nasal packings, ointments, lozenges, troches, candies, injectants, chewing gums, tablets, powders, sprays, injectants, powders, intravenous solution, and liquids.

192) The composition according to claim 190, wherein said composition further comprises a buffer that maintains pH of the composition at a range between about 4.0 and about 9.0.

193) The composition according to claim 192, wherein said buffer comprises a reducing reagent.

194) The composition according to claim 193, wherein said reducing reagent is dithiothreitol.

195) The composition according to claim 192, wherein said buffer comprises a metal chelating reagent.

196) The composition according to claim 195, wherein said metal chelating reagent is ethylenediaminetetracetic disodium salt.

197) The composition according to claim 192, wherein said buffer is a citrate-phosphate buffer.

198) The composition according to claim 192, further comprising a bactericidal or bacteriostatic agent as a preservative.

199) The composition according to claim 192, wherein said lytic enzyme is lyophilized.

200) The composition according claim 192, wherein said at least one lytic enzyme is present in a concentration of about 100 to about 500,000 active enzyme units per milliliter of fluid in the wet environment of the nasal or oral passages.

201) The composition according to claim 200, wherein said at least one lytic enzyme is present in a concentration of about 100 to about 50,000 active enzyme units per milliliter of fluid in the wet environment of the nasal or oral passages.

202) The composition according to claim 192, wherein said carrier is suitable for delivering said lytic enzyme parenterally.

203) The composition according to claim 192, wherein said lytic enzyme is produced by infecting said Streptococcus pneumoniae with a bacteriophage specific for said Streptococcus pneumoniae.

204) The composition according to claim 192, wherein said at least one lytic enzyme is produced by removing a gene for the lytic enzyme from the phage genome, putting said gene into a transfer vector, and cloning said transfer vector into an expression system.

206) The composition according to claim 204, wherein said transfer vector is a plasmid.

207) The composition according to claim 204, wherein said expression system is a bacteria.

208) The composition according to claim 190, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

209) The composition according to claim 204, wherein said expression system is a cell free expression system.

210) A composition for treating bacterial meningitis caused by Streptococcus pneumoniae, wherein said composition is formed by the method comprising the steps of:
i) obtaining an effective amount of at least one lytic enzyme genetically coded for by a bacteriophage specific for Streptococcus pneumoniae, wherein said at least one lytic enzyme specifically lyses the cell wall of said Streptococcus pneumoniae; and
ii) incorporating said lytic enzyme into a carrier which can deliver said lytic enzyme parenterally.

211) The composition according to claim 210, wherein said composition is administered parenterally.

212) The composition according to claim 210, wherein said composition is administered intravenously.

213) The composition according to claim 210, wherein said composition is administered subcutaneously.

214) The composition according to claim 210, wherein said composition is administered intramuscularly.

215) The composition according to claim 210, wherein said composition is administered intrathecally.

216) The composition according to claim 210, wherein said at least one lytic enzyme is produced by infecting said Streptococcus pneumoniae with a bacteriophage specific for said Streptococcus pneumoniae.

217) The composition according to claim 210, wherein said at least one lytic enzyme is produced by removing a gene for the lytic enzyme from the phage genome, putting said gene into a transfer vector, and cloning said transfer vector into an expression system.

218) The composition according to claim 217, wherein said transfer vector is a plasmid.

219) The composition according to claim 217, wherein said expression system is a bacteria.

220) The composition according to claim 210, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

221) The composition according to claim 220, wherein said expression system is a cell free expression system.

222) The composition according to claim 210, wherein said carrier is selected from the group consisting of distilled water, a saline solution, albumin, a serum, Ringer's solution, a buffered solution, a dextrose solution, and combinations thereof.

223) The composition according to claim 210, wherein said carrier comprises additives selected from the group consisting of p-hydroxybenzoates, stabilizers, fixed oils, ethyl oleate, neutral salts, dextrose, trehalose, dextrins, lactose, phosphate buffered saline, gelatin, albumin, vasoconstriction agents, organic acids organic acid salts, antioxidants, low molecular weight polypeptides, proteins, immunoglobulins, hydrophilic polymers, amino acids, monosaccharides, disaccharides, other carbohydrates including cellulose or its derivatives, glucose, chelating agents, sugar alcohols, counter-ions, non-ionic surfactants, glycerin, glycerol, DMSO, and combinations thereof.

224) A composition for treating eyes exposed to Streptococcus pneumoniae wherein said composition is formed by the method comprising the steps of:
a) obtaining an effective amount of at least one lytic enzyme genetically coded for by a bacteriophage specific for Streptococcus pneumoniae, wherein said at least one lytic enzyme specifically lyses the cell wall of said Streptococcus pneumoniae, and
b) incorporating said lytic enzyme into a carrier which can deliver said lytic enzyme to said eyes.

225) The composition according to claim 224, wherein said at least one lytic enzyme is produced by infecting said Streptococcus pneumoniae with a bacteriophage specific for said Streptococcus pneumoniae.

226) The composition according to claim 224, wherein said at least one lytic enzyme is produced by removing a gene for the lytic enzyme from the phage genome, putting said gene into a transfer vector, and cloning said transfer vector into an expression system.

227) The composition according to claim 224, wherein said transfer vector is a plasmid.

228) The composition according to claim 224, wherein said expression system is a bacteria.

229) The composition according to claim 224,wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

230) The composition according to claim 226, wherein said expression system is a cell free expression system

231) The composition according to claim 24, wherein said carrier is selected from the group consisting of eye wash solution and eye drop solution.

232) The composition according to claim 224, wherein said bacteriophage is selected from thegroupconsistingof Dp-1, DP-4, Cp-1, Cp-7, Cp-9, Cp-5, MM1, EJ-1,HB-3, HB-623, HB-746, ω-1, and ω-2.

233) The composition according to claim 224, wherein said bacteriophage is Dp-1.

234) A composition for treating ear infections, wherein said composition is formed by the method comprising the steps of:
a) obtaining an effective amount of at least one lytic enzyme genetically coded for by a bacteriophage specific for Streptococcus pneumoniae, wherein said at least one lytic enzyme specifically lyses the cell wall of said Streptococcus pneumoniae, and
b) inserting said at least one lytic enzyme in a suitable carrier for delivering said Streptococcus pneumoniae to an ear canal.

235) The composition according to claim 234, wherein said at least one lytic enzyme is produced by infecting said Streptococcus pneumoniae with a bacteriophage specific for said Streptococcus pneumoniae.

236) The composition according to claim 234, wherein said at least one lytic enzyme is produced by removing a gene for the lytic enzyme from the phage genome, putting said gene into a transfer vector, and cloning said transfer vector into an expression system.

237) The composition according to claim 236, wherein said transfer vector is a plasmid.

238) The composition according to claim 236, wherein said expression system is a bacteria.

239) The composition according to claim 234, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

240) The composition according to claim 236, wherein said expression system is a cell free expression system.

241) The composition according to claim 234, wherein said bacteriophage is selected from the group consisting of Dp-1, DP-4, Cp-1, Cp-7, Cp-9, Cp-5, MM1, EJ-1,HB-3, HB-623, HB-746, ω-1, and ω-2.

242) The composition according to claim 234, wherein said bacteriophage is Dp-1.

243) The composition according to claim 234, further comprising an antibiotic.

244) The composition according to claim 234, wherein said carrier comprises ingredients selected from the group consisting of vitamins, minerals, carbohydrates, sugars, amino acids, proteinacious materials, fatty acids, phospholipids, antioxidants, phenolic compounds, isotonic solutions, oil based solutions, oil based suspensions, and combinations thereof.

245) A contact lens solution by Streptococcus pneumoniae, wherein solution is formed by the steps of:
a) obtaining an effective amount of at least one lytic enzyme genetically coded for by a bacteriophage specific for said Streptococcus pneumoniae, wherein said at least one lytic enzyme specifically lyses the cell wall of said Streptococcus pneumoniae, and
b) incorporating said at least one lytic enzyme into a solution used for cleaning contact lenses.

246) The contact lens solution according to claim 245, wherein said at least one lytic enzyme is produced by infecting said Streptococcus pneumoniae with a bacteriophage specific for said Streptococcus pneumoniae.

247) The contact lens solution according to claim 245, wherein said at least one lytic enzyme is produced by removing a gene for the lytic enzyme from the phage genome, putting said gene into a transfer vector, and cloning said transfer vector into an expression system.

248) The contact lens solution according to claim 247, wherein said transfer vector is a plasmid.

249) The contact lens solution according to claim 247, wherein said expression system is a bacteria.

250) The contact lens solution according to claim 245, wherein said wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

251) The contact lens solution according to claim 245, wherein said expression system is a cell free expression system.

252) The contact lens solution according to claim 245, wherein said bacteriophage is selected from the group consisting of Dp-1, DP-4, Cp-1, Cp-7, Cp-9, Cp-5, MM1, EJ-1,HB-3, HB-623, HB-746, ω-1, and ω-2.

253) The contact lens solution according to claim 245, wherein said bacteriophage is Dp-1.

254) The contact lens solution according to claim 245, wherein said contact lens solution further comprising an antibiotic.

255) The contact lens solution according to claim 245, wherein said contact lens solution is an isotonic solution.

256) The contact lens solution according to claim 245, wherein said contact lens solution further comprises s odium chloride, sugar alcohols, borates, preservatives, and combinations thereof.

257) A method for the treating a lower respiratory tract illness caused by Streptococcus pneumoniae, comprising administering to a mouth, throat, nasal passage or lung of a mammal a composition comprising an effective amount of at least one lytic enzyme genetically coded for by a bacteriophage specific for Streptococcus pneumoniae, wherein said at least one lytic enzyme lyses the cell wall of said Streptococcus pneumoniae.

258) The method according to claim 257, wherein said composition further comprises a carrier selected from the group consisting of nasal sprays, nasal drops, nasal inhalants, nasal ointments, nasal washes, nasal injections, gels, nasal packings, ointments, lozenges, troches, candies, injectants, chewing gums, tablets, powders, sprays, injectants, powders, intravenous solution, and liquids.

259) The method according to claim 257, wherein said composition further comprises a buffer that maintains pH of the composition at a range between about 4.0 and about 9.0.

260) The method according to claim 259, wherein said buffer comprises a reducing reagent.

261) The method according to claim 260, wherein said reducing reagent is dithiothreitol.

262) The method according to claim 259, wherein said buffer comprises a metal chelating reagent.

263) The method according to claim 262, wherein said metal chelating reagent is ethylenediaminetetracetic disodium salt.

264) The method according to claim 259, wherein said buffer is a citrate-phosphate buffer.

265) The method according to claim 257, further comprising a bactericidal or bacteriostatic agent as a preservative.

266) The method according to claim 257, wherein said lytic enzyme is lyophilized.

267) The method according claim 257, wherein said at least one lytic enzyme is present in a concentration of about 100 to about 500,000 active enzyme units per milliliter of fluid in the wet environment of the nasal or oral passages.

268) The method according to claim 257, wherein said at least one lytic enzyme is produced by recombinant production from a nucleic acid that comprises a DNA having the sequence of bases 3687 to 4577 of SEQ ID No. 2 or a sequence that hybridizes with the complement of bases 3687 to 4577 of SEQ ID No. 2 under stringent hybridization conditions.

269) The method according to claim 259, wherein said composition is administered parenterally.

270) The method according to claim 257, wherein said lytic enzyme is produced by infecting said Streptococcus pneumoniae with a bacteriophage specific for said Streptococcus pneumoniae.

271) The method according to claim 257, wherein said at least one lytic enzyme is produced by removing a gene for the lytic enzyme from the phage genome, putting said gene into a transfer vector, and cloning said transfer vector into an expression system.

272) The method according to claim 271, wherein said transfer vector is a plasmid.

273) The method ac cording to claim 271, wherein said expression system is a bacteria.

274) The method according to claim 273, wherein said bacteria is selected from the group consisting essentially of E. coli and Bacillus.

275) The method according to claim 271, wherein said expression system is a cell free expression system
